(19) United States
(12) Patent Application Publication
(10) Pub. No.: US 2026/0224747 A1
(43) Pub. Date: Aug. 6, 2026

(54) GENE THERAPY MEDIATED ANGIOGENESIS TO ENHANCE SURVIVAL OF TRANSPLANTED FAT

(71) Applicant: Cornell University, Ithaca, NY (US)

(72) Inventors: Ronald G. Crystal, New York, NY (US); Jason A. Spector, New York, NY (US); Neil R. Hackett, New York, NY (US)

(21) Appl. No.: 19/152,346

(22) PCT Filed: Feb. 22, 2024
(86) PCT No.: PCT/US2024/016902

§ 371 Date: Jul. 30, 2025

Related U.S. Application Data
(60) Provisional application No. 63/486,323, filed on Feb. 22, 2023.

(51) Int. Cl. A61K 48/00; A61K 35/35; A61K 38/18; A61P 37/06; C12N 5/0775; C12N 15/86

(52) U.S. Cl. CPC A61K 48/005; A61K 35/35; A61K 38/1866; A61K 48/0083; A61P 37/06; C12N 5/0667; C12N 15/86; C12N 2510/00; C12N 2710/10343

(57) ABSTRACT

A method is provided comprising: administering to a mammal in need thereof a composition comprising isolated mammalian fat tissue infected with an adenovirus expressing one or more isoforms of VEGF as well as isolated mammalian fat tissue infected with an adenovirus expressing one, two or more isoforms of VEGF.

### CROSS-REFERENCE TO RELATED APPLICATIONS

This application claims the benefit of the filing date of U.S. application No. 63/486,323, filed on Feb. 22, 2023, the disclosure of which is incorporated by reference herein.

### INCORPORATION BY REFERENCE OF SEQUENCE LISTING

A Sequence Listing is provided herewith as a xml file, “1676193WO1.xml”, created on Feb. 19, 2024 and having a size of 38,849 bytes. The contents of the text file are incorporated by reference herein in their entirety.

### BACKGROUND

Autologous fat transplantation is a widely used surgical procedure to provide volume for soft tissue reconstruction (Firriolo et al., 2022; Liu et al, 2022). Because fat transplantation is a technically straightforward procedure, it is performed with minimal operator training and resource utilization. Clinical applications include correction of congenital deformities, traumatic wounds, soft-tissue loss, e.g., after oncologic surgery, skin grafting, scar and Dupuytren hand contractures, radiation damage, deformation from scleroderma and a variety of aesthetic applications such as breast, buttock and facial augmentation (Firriolo et al., 2022; Simonacci et al., 2017; Cao & Sheng, 2023). With minor modifications, autologous fat grafting is done in 3 steps: lipoaspiration, concentration of the fat and associated stromal vascular fraction and administration to the desired area (Simonacci et al., 2017; Bellini et al, 2017; Gause et al., 2014). While fat grafting is widely used for a variety of clinical reconstruction applications, the long-term results are often disappointing with reabsorption rates of 30-70% within a year (Simonacci et al., 2017; Bellini et al., 2017; Leong et al., 2005; Locke & de Chalain, 2008; Kakagia & Paula, 2014), primarily a result of ischemic necrosis of the transplanted adipocytes due to insufficient revascularization of the engrafted tissue.

The most common source of autologous transplanted fat is from the abdomen or thighs (Simonacci et al., 2017; Fontes et al., 2018). Ninety percent of the volume of transplanted fat is comprised of adipocytes, with the remainder made up of adipose stem cells, fibroblasts, endothelial cells and pericytes plus extracellular matrix (Charles-de-sa et al., 2015; Khouri & Khouri, 2017). The harvested fat is transplanted to the therapeutic site to: (1) provide volume for tissue reconstruction (e.g., post oncologic breast reconstruction); and/or (2) promote tissue regeneration and improve fibrosis (Khouri & Khouri, 2017; Toyserkani et al., 2017; Malik et al., 2020). A major challenge for the survival of transplanted fat is the initial lack of vascularity of the transplanted tissue, resulting in variable and often significant resorption of the transplant (Bellini et al., 2017; Auger et al., 2013; Shauly et al., 2022). Prior attempts to induce neoangiogenesis within the transplant from the surrounding tissue included adding vascular-related growth factors, such as insulin, insulin-like growth factors, vascular endothelial growth factor (VEGF) and platelet-rich plasma as well as a variety of cell-based therapies, including adipose stem cells alone, or genetically modified with VEGF165, or endothelial cells genetically modified to enhance survival (Liu et al., 2022; Shauly et al., 2022; Yi et al., 2007; Lu et al., 2009; Hamed et al., 2010). None of these strategies have reliably prolonged survival of transplanted fat likely because of insufficient bioavailability (in terms of quantity or time course) of the angiogenic factors needed to induce sufficiently vascularization (Major et al., 2022; Hu et al., 2022; Jiang et al., 2022; Dong et al., 2022).

### SUMMARY

The disclosure provides for the enhancement of the survival of transplanted fat using a gene therapy vector for angiogenic gene delivery, e.g., an adenovirus-based angiogenic gene therapy, to rapidly and efficiently induce vascularity into transplanted fat from the tissues surrounding the transplant.

The disclosure provides a method to induce angiogenesis, neovascularization, or vasculogenesis in a mammal, comprising: providing isolated mammalian cells or tissue comprising fat cells (explant), e.g., a mixture of cells including fat cells and optionally stromal vascular fraction (SVF); contacting the cells or tissue with a composition comprising a nucleic acid vector, e.g., a viral vector such as a non-integrating viral or non-viral vector, including for example an adenovirus vector, that expresses one or more proteins such as one or more isoforms of a protein, e.g., VEGFA, VEGFC, VEGFD, ANG1, ANG2, PDGFA, PDGFB, PLGF, TPO, HGF, FGF1, FGF2, TGFB, TNFA, CXCL2, CXCL8, CCL2, IL6, IL8, IL22, or EDN1, or a composition comprising one or more polypeptides, e.g., one or more isoforms of VEGFA, VEGFC, VEGFD, ANG1, ANG2, PDGFA, PDGFB, PLGF, TPO, HGF, FGF1, FGF2, TGFB, TNFA, CXCL2, CXCL8, CCL2, IL6, IL8, IL22, or EDN1; and transplanting the tissue having the nucleic acid composition or the composition comprising the one or more polypeptides and optionally SVF, into one or more sites in the mammal so as to induce angiogenesis, neovascularization, or vasculogenesis at, into or near the site of transplant. In one embodiment, blood vessel formation is induced into the transplanted tissue or transplanted cells. In one embodiment, the mammal is a human. In one embodiment, the fat tissue comprises mammalian adipocytes or adipose stem cells. In one embodiment, the fat cells are not cultured and/or expanded before transplant. In one embodiment, the vector expresses two or more isoforms of VEGF. In one embodiment, the vector expresses VEGF121. In one embodiment, the vector expresses VEGF165. In one embodiment, the vector expresses VEGF189. In one embodiment, the vector expresses VEGF121, VEGF165 and VEGF189, e.g., in a ratio of about 1:1:1 or VEGF189>VEGF165>VEGF121. In one embodiment, the composition comprises two or more isoforms of VEGF. In one embodiment, the composition comprises VEGF121. In one embodiment, the composition comprises VEGF165. In one embodiment, the composition comprises VEGF189. In one embodiment, the composition comprises VEGF121, VEGF165 and VEGF189, e.g., in a ratio of about 1:1:1 or VEGF189>VEGF165>VEGF121. In one embodiment, liquid is removed from the mammalian tissue or cells prior to contacting with the vector or polypeptide(s). In one embodiment, the vector is a non-integrating viral vector, e.g., adenovirus. In one embodiment, the adenovirus is E1⁻ and/or E3⁻. In one embodiment, the adenovirus is a human adenovirus. In one embodiment, the adenovirus is a non-human primate adenovirus. In one embodiment, the transplanted cells secrete VEGF. In one embodiment, at least two portions of the tissue or cells contacted with the composition are transplanted at the same site. In one embodiment, at least two portions of the tissue or cells contacted with the composition are transplanted at different sites. In one embodiment, the fat tissue is obtained from a thigh, abdomen, or buttock of the mammal. In one embodiment, the portion is transplanted to a breast, buttock, face, abdomen, or hand of the mammal. In one embodiment, the fat tissue is obtained from or transplanted to the head, trunk, abdomen, genitalia, or extremities of a mammal. In one embodiment, the composition is a sustained release composition comprising one or more proteins that induce neovascularization, angiogenesis or vasculogenesis. In one embodiment, transplanted tissue or cells contacted with a nucleic vector encoding isolated polypeptide(s) releases the one or more polypeptides beginning at about 4 to 8 hours and up to 14 to 21 days following transplant. In one embodiment, a sustained release composition comprising isolated polypeptide(s) releases the one or more polypeptides beginning at about 4 to 8 hours and up to 14 to 21 days following transplant. In one embodiment, after contact with a vector, the fat may be injected in a standard fan shaped pattern to release scar tissue, e.g., 1×10⁶ to 1×10⁸ cells per injection, or per 0.1 mL, 1 mL, 5 mL, 10 mL, or 50 mL or more. The volume of fat injected may vary, e.g., from 0.5 to 25 mL, e.g., 1 mL to 5 mL, 5 mL to 10 mL or 10 mL to 15 mL including 1 mL, 5 mL, 10 mL, 50 mL, 100 mL, 150 mL, 200 mL, 250 mL, 300 mL, 350 mL, 400 mL, 450 mL or 500 mL or more.

Also provided is a method, comprising: providing isolated mammalian tissue comprising fat cells or mammalian cells that include fat cells and optionally a stromal vascular fraction (SVF) which may include mesenchymal progenitor/stem cells, preadipocytes, endothelial cells, pericytes, T cells, M2 macrophage, or any combination thereof; and contacting the tissue with a composition comprising a nucleic acid vector expressing one or more isoforms of a proteins such as VEGF so that the fat cells comprise the nucleic acid, e.g., extrachromosomally, or with a composition comprising a sustained release formulation comprising one or more polypeptides such as one or more isoforms of VEGF, e.g., beginning at about 4 to 8 hours and up to 14 to 21 days following transplant. In one embodiment, the mammal is a human. In one embodiment, the tissue comprises mammalian adipocytes or adipose stem cells, or both. In one embodiment, the vector expresses or composition comprises two or more isoforms of VEGF. In one embodiment, the vector expresses or composition comprises VEGF121. In one embodiment, the vector expresses or composition comprises VEGF165. In one embodiment, an adenovirus expresses or composition comprises VEGF189. In one embodiment, an adenovirus expresses or composition comprises VEGF121, VEGF165 and VEGF189. In one embodiment, the ratio of the VEGF isoforms is about 1:1:1. In one embodiment, the ratio of the isoforms is VEGF189>VEGF165>VEGF121. In one embodiment, liquid is removed from the mammalian tissue prior to contacting. In one embodiment, the vector is an adenovirus, e.g., the adenovirus is E1⁻ and/or E3⁻, a human adenovirus or a non-human primate adenovirus. In one embodiment, the adenovirus is serotype 5, 6, 26, 35 or 36. In one embodiment, the dose of the adenovirus is at least 2×10⁹, 5×10⁹ or 1×10¹⁰ gc/0.1 mL.

Also provided is isolated infected mammalian fat tissue or a mixture of adipocytes and at least one other cells type prepared by the methods.

Further provided is a composition comprising isolated mammalian fat tissue or fat cells genetically modified to express one or more polypeptides, e.g., one or more isoforms of VEGF. In one embodiment, the composition has at least 1 g to 10 g, 10 g to 25 g, 25 g to 50 g, 50 g to 100 g, 100 g to 500 g of the isolated mammalian tissue, e.g., per 0.1 mL, 1 mL, 5 mL, 10 mL, 50 mL, 100 mL, 150 mL, 200 mL, 250 mL, 300 mL, 350 mL, 400 mL, 450 mL or 500 mL or more. In one embodiment, the composition has at least 1×10⁴ to 1×10⁶ cells, at least 1×10⁶ to 1×10⁸ cells, at least 1×10⁸ to 1×10¹⁰ cells, at least 1×10¹⁰ to 1×10¹² cells, at least 1×10¹² to 1×10¹⁴ cells or at least 1×10¹⁴ to 1×10¹⁶ cells, e.g., present in the SVF.

Also provided is a composition comprising isolated mammalian fat tissue or fat cells comprising a nucleic acid vector encoding one or more polypeptides and a sustained release formulation, e.g., comprising nanoparticles or microparticles, comprising one or more polypeptide(s), e.g., one or more isoforms of VEGF, which polypeptide(s) are released beginning at about 4 to 8 hours and up to 14 to 20 days following transplant. In one embodiment, the composition or formulation has at least 1 g to 10 g, 10 g to 25 g, 25 g to 50 g, 50 g to 100 g, 100 g to 500 g of the isolated mammalian tissue or fat cells. In one embodiment, the composition or formulation has at least 1×10⁴ to 1×10⁶ cells, at least 1×10⁶ to 1×10⁸ cells, at least 1×10⁸ to 1×10¹⁰ cells, at least 1×10¹⁰ to 1×10¹² cells, at least 1×10¹² to 1×10¹⁴ cells or at least 1×10¹⁴ to 1×10¹⁶ cells. In one embodiment, the formulation has at least 1 μg to 10 μg, 10 μg to 25 μg, 25 μg to 50 μg, 50 μg to 100 μg, 100 μg to 500 μg of the particles or the polypeptide(s). In one embodiment, the formulation has at least 1 mg to 10 mg, 10 mg to 25 mg, 25 mg to 50 mg, 50 mg to 100 mg, 100 mg to 500 mg of the particles or the polypeptide(s). In one embodiment, the formulation has at least 1 ng to 10 ng, 10 ng to 25 ng, 25 ng to 50 ng, 50 ng to 100 ng, 100 ng to 500 ng of the particles or the polypeptide(s).

Further provided is a method to augment a void (tissue deficiency) or contour a deformity, e.g., augment soft tissue deficiency or deformity, enhance skin grafting, alter scarring, improve split thickness or full thickness skin grafting, or augment the volume or shape of a body part in a mammal, comprising: administering to a mammal a composition comprising isolated mammalian fat tissue genetically modified to exogenously express one or more isoforms of a protein such as VEGF or a composition comprising isolated mammalian fat tissue and a nanoparticles or microparticles comprising one or more proteins, e.g., one or more isoforms of VEGF. In one embodiment, the mammal is a human. In one embodiment, the void (tissue deficiency) is due to surgery. In one embodiment, the mammal has congenital deformities, traumatic wounds, soft-tissue loss after oncologic surgery, is in need of skin grafting, has one or more scars, has Dupuytren hand contractures, has radiation damage, or has deformation from scleroderma. In one embodiment, the composition is administered to a breast, buttock or face of the mammal. In one embodiment, the fat tissue is obtained via lipoaspiration. In one embodiment, the fat tissue comprises adipose stem cells and optionally fibroblasts, endothelial cells and/or pericytes. In one embodiment, the fat and associated stromal vascular fraction is administered, e.g., injected, to a desired area.

In one embodiment, utilizing transient expression of one or more VEGF isoforms, results in an induction of angiogenesis. In one embodiment, utilizing transient expression of 2 or more, VEGF isoforms, e.g., 3 of the major isoforms of VEGF, may result in an induction of angiogenesis that is 10-100-fold more potent than any one isoform. This strategy should provide a sufficient angiogenic stimulus to induce vascularization of transplanted fat and significantly enhance the survival of the transplant.

### BRIEF DESCRIPTION OF FIGURES

FIG. 1. Ad5VEGF-AII or Ad5VEGF-AII6A+ induced vascularization of transplanted autologous fat. The therapy is administered to the harvested fat which is then transplanted. In vivo, the genetically modified fat secretes all 3 major VEGF isoforms (121, 165 and 189) which rapidly induces new blood vessel growth into the transplanted fat from the surrounding tissue, resulting in increased fat survival. Two Ad vectors are disclosed, Ad5VEGF-AII and Ad5VEGF-AII6A+ (see Whitlock et al. (2004) and Amano et al. (2005), the disclosures of which are incorporated by reference herein).

FIGS. 2A-2B. Adenovirus-mediated expression of β-galactosidase in adipose tissue. Shown is the blue staining of β-galactosidase protein expression 48 hours following administration in vivo of the AdCMV.βgal to rat retroperitoneal adipose tissue. Dose 109 pfu. Magnification, 10×. A) Naive. B) AdCMV.βgal.

FIGS. 3A-3C. AdCMV.VEGF-mediated neovascularization of fat in vivo. An Ad5 vector coding for VEGF165 (109 pfu) was administered in vivo to rat retroperitoneal adipose tissue. A) Adipose tissue VEGF levels over time. B) Adipose tissue gross vessel count over time. C) Quantification of adipose tissue capillary number over time, AdCMV.VEGF (●) vs control AdCMV.Null (◯), sham (□).

FIGS. 4A-4F. AdCMV.VEGF induction of vascularity in rat retroperitoneal adipose tissue 30 days following administration. A) Naive. B) AdCMV.VEGF. Magnification 30×. C-F. Histologic evaluation of neovascularization following local administration of AdCMV.VEGF. Doses 109 pfu. C) Naive. D) AdCMV.Null (control). E-F) AdCMV.VEGF. Panels stained with α-actin. Magnification, 400×.

FIG. 5. Dose-response of an Ad5βgal vector mediating β-galactosidase in harvested human fat in vitro. The doses of Ad5βgal were administered to the fat with subsequent incubation at 37° C. for 14 hours. Shown is β-galactosidase activity in the human harvested fat mediated by increasing doses of Ad5βgal. The AdNull vector with no transgene serves as a control.

FIGS. 6A-6B. Experimental models. To test whether enhanced angiogenesis improves the performance of a fat transplant, human fat lipoaspirate from donors is mixed with either vehicle (PBS), a control adenovirus vector that does not express a transgene (Ad5Null), or an adenovirus vector expressing the VEGFAII6A+ chimeric multi-isoform transgene. A) In vitro model. The mixture of lipoaspirate and vector is incubated at 37° C. for 24 hr and is then assessed to determine levels of VEGF mRNA and protein expression in each sample. B) In vivo model. The mixture of lipoaspirate and vector is used to create a subdermal transplant. At time points ranging from 7 to 90 days, the transplant is harvested and assessed for VEGF mRNA and protein expression, gross morphology, histology, CD31 immunohistochemical staining and transplant volume and density via microCT.

FIG. 7. VEGF mRNA expression assessed using the in vitro model of gene transfer to human lipoaspirate. Human lipoaspirate was mixed with a range of doses of Ad5VEGFAII6A+ and compared with samples mixed with either Ad5Null (10¹⁰ gc) or PBS as described in FIG. 6. After 24 hr, VEGF mRNA was assessed using quantitative RT-PCR.

FIG. 8. VEGF protein expression assessed in the in vitro model of gene transfer to human lipoaspirate. Human lipoaspirate was mixed with a range of doses of Ad5VEGFAII6A+ and compared with samples mixed with either Ad5Null (10¹⁰ gc) or PBS as described in FIG. 6. After 24 hr, VEGF protein was assessed using an ELISA assay.

FIG. 9. VEGF mRNA expression assessed using the in vivo model of gene transfer to human lipoaspirate. Human lipoaspirate was mixed with doses of Ad5VEGFAII6A+ ranging from 10⁹ to 10¹⁰ gc and compared with samples mixed with either Ad5Null (10¹⁰ gc) or PBS as described in FIG. 6. After 7 days, VEGF mRNA was purified from transplanted fat and assessed using quantitative RT-PCR.

FIG. 10. VEGF protein expression assessed in the in vivo model of gene transfer to human lipoaspirate. Human lipoaspirate was mixed with Ad5VEGFAII6A+ (10⁹, 4×10⁹, or 10¹⁰ gc) and compared with samples mixed with either Ad5Null (10¹⁰ gc) or PBS as described in FIG. 6. After 7 days, fat transplants were recovered and VEGF protein was assessed using an ELISA assay.

FIG. 11. Gross angiogenesis morphology in mice receiving fat transplants. Seven days after placement of fat transplants in the Nu/j mouse, mice were sacrificed. Upon dissection of the transplant, enhanced angiogenesis could be observed in the mucosal surface of the skin that was associated with the transplant (left panels above). Upon removal of the transplants, transplant-associated blood vessels could be observed (right panels above). Mice that received a high dose of Ad5VEGFAII6A+ exhibited more transplant-associated blood vessels as indicated by the pink coloration of the transplant surfaces.

FIG. 12. Quantitative assessment showing time course of endothelial-specific CD31+ immunohistochemistry in fat transplants. Mice were sacrificed at 7, 14, 30 or 90 days after placement of fat transplants. After recovery of the transplants, tissue was fixed and processed for paraffin sectioning. Sections were stained for CD31 and visualized via DAB precipitation. The QuPath image analysis program was used to assess the % area of CD31 staining as a percentage of total transplant area.

FIG. 13. Quantitative microCT assessment showing time course of changes in transplant density. Mice were sacrificed at 7, 14, 30 or 90 days after placement of fat transplants. After recovery of the transplants, tissue was evaluated via microCT. The density of the transplant was measured in Hounsfield units (HU). Note that the change in density is not significant for all Ad5VEGFAII6A+ conditions.

FIG. 14. Quantitative microCT assessment showing time course of changes in transplant volume. Mice were sacrificed at 7, 14, 30 or 90 days after placement of fat transplants. After recovery of the transplants, tissue was evaluated via microCT. The volume of the transplant was measured in mm³. Note that the only condition that did not experience a statistically significant decrease in transplant volume between days 30 and 90 was the high dose Ad5VEGFAII6A+ treatment.

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense, and the scope of the present invention is defined by the appended claims.

Autologous fat grafts are widely used to help reconstruct soft tissue deficits resulting from congenital deformations, traumatic wounds, oncologic surgery, skin grafts, scar contracture, radiation damage and a variety of aesthetic applications (Simonacci et al., 2017; Gir et al., 2012). The long-term results are often disappointing because of variable loss of the fat graft, with resorption rates of up to 70% within 1 year (Simonacci et al., 2017; Bellini et al., 2017; Leong et al., 2005; Locke & de Chalain, 2008; Kakagia & Pallua, 2014) due to inadequate vascularization of the transplant (Auger et al., 2013; Shauly et al., 2022; Harris et al., 2019). Many strategies have been tried to stimulate vascularization of transplanted fat to enhance transplant survival in animal models and human trials, but the results were unsatisfactory or inconsistent.

Strategies to augment retention of fat transplants follow several distinct strategies, generally falling into one of two categories: either one or more cell types from the fat or from an exogenous source is included with the lipoaspirate in a more or less characterized form, or (2) a biologic therapeutic entity, such as a scaffold material, signaling molecule, or a gene transfer vector, is combined with the lipoaspirate in hopes of increasing retention of fat. The field of augmenting fat transfer has been reviewed recently (Liu et al., 2023).

The simplest intervention for the surgeon is to manipulate the material available from surgery. Isolation of a stromal vascular fraction (SVF) is achieved by tissue disruption followed by differential centrifugation to collect a mixed cell fraction that includes adipocyte stem cells, endothelial cells and/or endothelial precursor cells, pericytes, immune cells, and other stromal cells. The SVF is then mixed with conventional lipoaspirate prior to transplantation. Among human studies that have employed SVF, reported volume retention at >12 months ranges from 47 to 69% (Liu et al., 2023). While several studies showed a significant improvement in the presence of SVF, additional studies sought to find the component of SVF that had the greatest bioactivity. One group utilized autologous adipocyte stem cells (ADSC) achieving up to 80% volume retention at 18 months (Kølle et al., 2020), a laudable result complicated only by the fact that it requires two full-sedation procedures separated by 3 weeks, thus increasing the risk of the solution, and leading to a 25% dropout rate in the ADSC treatment group. The highest retention rate reported in a clinical trial (>90%) made use of a modification of the SVF procedure that purportedly utilized a rapid intraoperative acquisition of ADSCs from the patient's fat, however, those results have been questioned (Gontijo et al., 2017; Swanson, 2018; Gontijo et al., 2018). Using a different cell-enhanced transplant strategy, Dong et al. (2023) showed that supplementation of lipoaspirate with immortalized endothelial cells could improve volume retention at 6 months after transplant. The challenge of obtaining FDA approval for a transplant strategy that included delivery of immortalized cells would be significant.

In the field of fat transplants, gene transfer strategies have not progressed as far as cell-assisted methods have. Yi et al. (2007) employed an expression cassette encoding only one VEGF isoform (VEGF165) without reporting the serotype of adenovirus. The experiment was concluded after 3.5 months, before the full course of transplant regeneration had occurred. Most problematic, the VEGF levels reported in mouse serum were highest at Day 1 after transplant and dropped continuously thereafter, a profile unlike any other in vivo adenovirus-mediated gene transfer. Lu et al. (2009) transfected ADSCs with a plasmid expressing VEGF before transferring the ADSCs along with a conventional fat transplant into nude mice. The genetically modified ADSCs provided over 70% volume retention at 6 months after transplant. Finally, a similar concept was tested by Jun-Jiang and Huan-Jiu (2016) using adenoviruses to transfer a VEGF165 gene to ADSCs.

The disclosure provides methods for the enhancement of the survival of transplanted fat using a gene therapy vector for angiogenic gene delivery, e.g., an adenovirus-based angiogenic gene therapy, or a composition having a release profile for one or more polypeptide(s), to rapidly and efficiently induce vascularity into transplanted fat from the tissues surrounding the transplant.

There are three zones associated with a transplant (Shih et al., 2020). The outermost zone is the “surviving zone” that is located within 300 microns of the surface of the transplant In the surviving zone, adipocytes, adipocyte stem cells, and endothelial cells derive support from surrounding healthy tissues and will survive. The next zone is the “regenerating zone,” that is located between 600 and 1,200 microns from the surface of the transplant. In the regenerating zone, adipocytes that are sensitive to ischemia die while adipocyte stem cells survive and give rise to a new generation of adipocytes that associate with new blood vessels to form stable adipose tissue. Finally, the “necrotizing zone” is located greater than 1,200 microns from the transplant surface. In the necrotizing zone, both adipocytes and adipose stem cells die. Removal of dead adipocytes by macrophages takes weeks to months, accounting for the initial stability in the volume of the transplant. A timeline of up to 1 year likely establishes a stable tissue. For these reasons, plastic surgeons attempt to add adipose tissue in thin layers rather than in a bolus. If the size of the regenerating zone can be increased at the expense of the necrotizing zone, then the thickness of fat transplants may be increased and fewer procedures may be needed to accomplish a desirable outcome. The use of the disclosed vectors likely results in enhancement of angiogenesis that extends the depth of the regenerating zone.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene therapy, and may encompass targeting, binding, uptake, transport, localization, replicon integration and expression.

“Gene therapy” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical examples of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators) and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less or 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of (e.g., framework sequence(s) or CDR sequence(s)) a reference polypeptide sequence, e.g., they have at least 80%, 85%, 90%, 91%, 92%, 93%, 94%, 95%, 96%, 97%, 98%, 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, at least 90 to 95 percent sequence identity, at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides.

Exemplary Harvesting and Processing Methods for Tissue or Cells Prior to Contact with a Gene Delivery Vector or Protein Delivery Vehicle

Autologous fat grafting (AFG) has long been used as an esthetic technique for correcting volume loss or contour defects. The standard AFG procedure used is the Coleman technique, which may be subdivided into harvesting, refinement, and application steps. Fat harvesting sites are selected according to accessibility or esthetic factors, with studies showing similar outcomes between different donor regions. Hand-held syringe aspiration, suction-assisted lipectomy, and ultrasound-assisted lipectomy may be employed.

For example, small incisions are made, and a blunt-tipped harvesting cannula is advanced into the donor region. Fat may be harvested from the abdomen, e.g., upper abdomen and/or lower abdomen, the flank, buttocks, breast, knee, hip, thigh, e.g., lateral thigh, and inner thigh, and/or calf, e.g. using liposuction. Tumescent anesthesia has benefits, including reduced pain, reduced blood loss, and improved ease of fat removal. Tumescent solution, containing saline with local anesthetic and/or adrenaline, may be infiltrated locally to ease aspiration and minimize bleeding, Various adipose tissue processing techniques may optionally be employed, such as centrifugation, gravity separation, washing, and/or filtration. For example, harvested lipoaspirate is then optionally processed by centrifugation to obtain a condensed adipose tissue pellet or subjected to methods to remove excess fluid. The lipoaspirate product is exposed to the gene delivery vehicle or protein containing particles, e.g., for 5 up to 20 to 30 minutes, prior to administration, e.g., via injection, into one or more recipient sites.

Although the Coleman technique represents the standard AFG technique, several variations exist. One of these is cell-assisted lipotransfer (CAL). In CAL, either purified adipocytes and/or adipocyte stem cells or the mixed cellular components of the stromal vascular fraction (SVF) are added to lipoaspirate tissue before application. Alternatively, the SVF or isolated ADSCs may be injected without reconstitution; here, the intention is to provide equivalent regenerative effects while limiting the volume of fat injected.

In one embodiment, telfa rolling is employed.

In one embodiment, the site is treated with lidocaine or lidocaine/epinephrine, cells are harvested, and optionally processed by centrifugation at 300 to 500 g for 5 minutes or 1000 to 1500 g for 3 to 10 minutes.

In one embodiment, the site is treated with bupivacaine, mepivacaine, prilocaine ropivacaine, articaine/epinephrine, or lidocaine and then cells are harvested.

In one embodiment, a hand-held syringe is employed to harvest cells, e.g., via aspiration.

In one embodiment, suction assisted lipectomy, e.g., −15 inH to −25 inHg or 400 mmHg to 450 mmHg, is employed.

In one embodiment, ultrasound assisted lipectomy is employed.

In one embodiment, a tumescent solution is employed.

In one embodiment, a 2 mm, 3 mm, 4 mm, 5 mm or 6 mm aspiration cannula is employed.

In one embodiment, hand-held syringe aspiration is employed followed by ex vivo tumescent solution infiltration and liposuction and gravity separation.

In one embodiment, a tumescent solution and hand-held syringe aspiration is employed, optionally followed by collagenase treatment.

In one embodiment, one or more enzymes are combined, e.g., with the explanted fat tissue.

In one embodiment, suction assisted lipectomy or hand-held syringe aspiration is performed at −25 in Hg or hand-held syringe aspiration followed by centrifugation at 200 g, 500 g for 2 minutes.

In one embodiment, a tumescent solution and hand-held syringe aspiration are employed followed by gravity separation, centrifugation at 3000 rpm (6000 g) for 3 minutes, or manual washing with saline/centrifugation at 3000 rpm (6000 g) for 3 minutes.

In one embodiment, a tumescent solution and suction assisted lipectomy are employed followed by gravity separation or centrifugation at 3000 rpm for 3 minutes.

In one embodiment, a tumescent solution and suction-assisted liposuction are employed followed by gravity separation for 20 minutes, centrifugation at 3000 rpm (1200 g) for 3 minutes, or machine washing/filtering.

In one embodiment, a tumescent solution and hand-held syringe aspiration are employed followed by centrifugation at 1500 rpm for 3 minutes or cotton gauze rolling with large pieces of nonadherent dressing for 30 seconds and optional collagenase treatment.

In one embodiment, a tumescent solution and hand-held syringe aspiration are employed followed by centrifugation at 1800 g for 3 minutes and gauze filtration for 3 minutes.

In one embodiment, a tumescent solution and hand-held syringe aspiration are employed followed by centrifugation at 1500 rpm for 5 minutes or towel processing by placing lipoaspirates on a towel or absorbant pad to remove fluid, oil, and/or debris.

In one embodiment, a tumescent solution and hand-held syringe aspiration or suction assisted lipectomy are employed followed by centrifugation at 500 g for 2 minutes, washing with lactated Ringer solution, washing with 0.9% saline, washing with lactated Ringer solution and centrifugation, or washing with normal saline and centrifugation.

In one embodiment, a tumescent solution and hand-held syringe aspiration are employed followed by centrifugation and cotton gauze rolling, and suction assisted lipectomy was performed for filtering, followed by an optional centrifugation at 3000 rpm (1200 g) for 3 minutes, cotton gauze processing with large pieces of nonadherent dressing, or filtration.

In one embodiment, a tumescent solution and hand-held syringe aspiration are employed followed by gravity separation for 15 minutes, centrifugation at 1256 g for 3 minutes, and washing with saline.

In one embodiment, a tumescent solution and hand-held syringe aspiration are followed by gravity separation or centrifugation at 3600 rpm for 3 minutes.

In one embodiment, after hand-held syringe aspiration, the harvested tissue is subjected to centrifugation at 3400 rpm for 3 minutes or washed with saline.

In one embodiment, a tumescent solution and hand-held syringe aspiration are followed by centrifugation at 3000 rpm for 3 minutes or washing/filtering.

In one embodiment, approximately 300 mL lipoaspirate from either thighs or abdomen may be obtained using water-jet-assisted liposuction (body-jet, Human med AG, Schwerin, Germany, http://www.humanmed.com/en) is obtained.

In one embodiment, harvested lipoaspirate is centrifuged at 3000-3500 rpm for 1-4 minutes.

Any device may be employed, e.g., after harvesting, to concentrate fat cells, see, e.g., https://www.allerganaesthetics.com/brands/revolve, and https://sientra.com/for-us-surgeons/viality/

### Gene Therapy Vectors

The disclosure provides a gene therapy vector comprising a nucleic acid sequence which encodes one or more proteins, e.g., one or more isoforms of VEGF. The disclosure further provides a method of using the vector to enhance vascularization of tissue adjacent to and including the gene therapy vector transfected or infected cells after those cells are transplanted. Various aspects of the gene therapy vector and method are discussed below. Although each parameter is discussed separately, the gene therapy vector and method comprise combinations of the parameters set forth below to evoke neovascularization. Accordingly, any combination of parameters can be used according to the gene therapy vector and the method.

A “gene therapy vector” is thus any molecule or composition that has the ability to carry a heterologous nucleic acid sequence into a suitable host cell where synthesis of the encoded protein takes place. Typically, a gene therapy vector is a nucleic acid molecule that has been engineered, using recombinant DNA techniques that are known in the art, to incorporate the heterologous nucleic acid sequence. Desirably, the gene therapy vector is comprised of DNA. Examples of suitable DNA-based gene therapy vectors include plasmids and viral vectors. However, gene therapy vectors that are not based on nucleic acids, such as liposomes, are also known and used in the art. The gene therapy vector can be based on a single type of nucleic acid (e.g., a plasmid) or non-nucleic acid molecule (e.g., a lipid or a polymer). The gene therapy vector can be integrated into the host cell genome, or can be present in the host cell in the form of an episome.

Gene delivery vectors within the scope of the disclosure include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

In one embodiment, the gene therapy vector is a viral vector. Suitable viral vectors include, for example, retroviral vectors, lentivirus vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Thus, the viral vector can be any suitable viral vector. Suitable viral vectors include, but are not limited to, reoviruses, adenoviruses, adeno-associated viruses, papovaviruses, parvoviruses, picornaviruses, and enteroviruses of any suitable origin (e.g., of animal origin (e.g., avian or mammalian) and desirably of human origin). Other suitable viral vectors are known in the art and are well characterized. Examples of such viral vectors are described in, for example, Fields et al., VIROLOGY Lippincott-Raven (3rd ed. (1996) and 4th ed. (2000)); ENCYCLOPEDIA OF VIROLOGY, R. G. Webster et al., eds., Academic Press (2nd ed., 1999); FUNDAMENTAL VIROLOGY, Fields et al., eds., Lippincott-Raven (3rd ed., 1995); Levine, “Viruses,” Scientific American Library No. 37 (1992); MEDICAL VIROLOGY, D. O. White et al., eds., Academic Press (2nd ed. 1994); and INTRODUCTION TO MODERN VIROLOGY, Dimock, N. J. et al., eds., Blackwell Scientific Publications, Ltd. (1994). T viral vector may be derived from, or based on, a virus that normally infects animals, such as mammals (such as humans). Adenoviral (Ad) vectors based on human or non-human primate adenoviruses may be used as viral vectors.

In one embodiment, the gene therapy vector is a non-integrating viral vector, e.g., Ad, AAV, integration-deficient lentiviral vectors (IDLVs), poxviral and others.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

In one embodiment, lentiviral vectors may be employed. These vectors do not encode any viral product, as the viral proteins are provided in trans from several packaging plasmids to split the original viral genome. Accessory genes, often responsible for pathogenic features, have been progressively removed from the production system. Vectors have also been made self-inactivating (SIN) by deleting the transcriptional promoter/enhancer from the 3′ LTR in the transfer plasmid; this deletion is copied onto the 5′ end of the vector during the reverse transcription cycle, abolishing expression from the viral LTR. SIN vectors are therefore dependent on an internal promoter to provide transgenic expression40. Additionally, high-efficiency lentiviral transduction can be achieved with IDLVs, produced through the use of integrase mutations that specifically prevent proviral integration, resulting in the generation of increased levels of circular vector episomes. Lacking replication signals, lentiviral episomes mediate transient transduction in dividing cells and stable expression in quiescent cells. It is also possible to use retroviral vectors for so-called retrovirus particle-mediated mRNA transfer (RMT), whereby vector mutants unable to start reverse transcription are instead transiently translated, and lentiviral vectors for protein delivery.

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. In addition, adenoviral vectors can be produced at very high titers, allowing efficient gene therapy with small volumes of virus.

Adenovirus vector features include efficient delivery to dividing and non-dividing cells, retention as non-integrated nuclear linear episomes, high but transient, and a capacity of ~8 kb.

Adenoviruses are a family of DNA viruses with an icosahedral, 70-100 nm in diameter, non-enveloped capsid engulfing a double-stranded (ds) DNA genome. These viruses can infect quiescent and dividing cells and replicate in the cell nucleus. Human Ad serotypes from a range of >50 Ad subdivisions/clades, with a typical Ad5 vector genome of ~36 kb encoding genes that are expressed before (Early, E) and after (Late, L) viral replication. Early transcription units encode proteins required for viral transactivation and host-virus interactions. Non-human primate (NHP) adenoviruses from chimpanzees, bonobos and gorillas and various other species may be employed in the methods. Exemplary non-human including simian, e.g., gorilla, chimpanzee, and rhesus, adenoviruses include but are not limited to GC44, GC45, GC46, Pan5, Pan6, Pan7, Pan9, GRAd, AdC7, AdC21, AdC6, SAdV-11, SAdV-16, PanAd3, ChAd23, ChAd24, sAd16, sAd19, ChAdOx1, AdC68 sAd33, RhAd51, RhAd52 or RhAd53, as well as adenoviruses disclosed in Abbink et al. (J. Virol., 89:1512 (2015)), the disclosure of which is incorporated by reference herein. Conventional Ad vectors are constructed by substituting the E1 region of the adenovirus genome with the transgene cassette of interest [E1−].

### Adeno-Associated Virus Vectors

Recombinant adeno-associated viruses (rAAV) are derived from nonpathogenic parvoviruses, and produce transgene expression lasting months to years in most systems. Moreover, like adenovirus, adeno-associated virus vectors also have the capability to infect replicating and nonreplicating cells and are believed to be nonpathogenic to humans.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

Poxviral vectors, including vaccinia Poxviral vectors features include large-capacity dsDNA viruses (>25 kb of foreign DNA) and transient expression of proteins. Poxviruses are members of the family Poxviridae. They are dsDNA viruses about 200-400n min length with a genome of about 190 kb, which is flanked by ~10 kb ITRs, and exist in two forms: an intracellular naked virion (INV) and an extracellular enveloped virion (EEV). Transcription and DNA replication occur in the cytoplasm, where the progeny DNA is generated by the synthesis and resolution of large concatemeric molecules. Recombinant poxviruses have the transgene of interest commonly inserted by homologous recombination and driven by a poxviral promoter rather than a constitutive viral or mammalian promoter, since they are cytoplasmatic viruses and encode their own RNA polymerase. Modified Vaccinia virus Ankara (MVA) is licensed as third-generation vaccine against smallpox. Recombinant MVAs (rMVAs) can be used for protein production and as vaccines against infectious diseases, cancer and other pathologies64.

Other non-integrating viral vector systems include herpes virus vectors, and particularly those based on HSV-1.

Sendai virus, an RNA virus with no risk of genomic integration that can infect a wide range of cell types including HSPC, may also be employed.

Other DNA molecules for gene delivery include minicircle DNA, mini-intronic plasmids and closed-ended linear duplex (CELiD). In addition to DNA, mRNA can also be delivered using non-viral vectors to provide short-term transgene expression. In the case of mRNA, it only needs to enter the cytoplasm to function, nuclear entry is removed as a significant barrier to function. The transient expression obtained with mRNA delivery is useful where expression is needed for only a short period of time. A number of non-viral methods for nucleic acid delivery have been developed, which can be classified as physical or chemical. Physical methods include the use of ultrasound or electrical currents to temporarily increase the permeability of target cells (sonoporation and electroporation, respectively), direct injection of DNA into single cells, ballistic propulsion of DNA-coated particles and hydrodynamic gene delivery involving the rapid injection of a large volume of DNA solution (8-10% of body weight). Gene delivery by physical methods is fairly simple but offers no protection from nucleases for the nucleic acid.

In contrast, chemical carriers typically encapsulate nucleic acids thereby protecting the payload from nucleases. Chemical gene delivery vectors usually employ a cationic species to condense the anionic nucleic acids and in the process form nanoparticles for delivery. Cationic liposomes have been extensively studied and are among the most widely used non-viral vectors. Later, addition of cationic polymers (producing so-called lipopolyplex) was shown to enhance gene delivery. Mechanistically, the liposome likely provides the mechanism for endosomal escape whilst the polymer enables efficient condensation and packaging of the nucleic acid therefore forming small, stable, discrete and homogenous nanoparticles. Further attempts at improving non-viral formulations have been made with the addition of components to improve bioavailability in vivo through shielding of complexes using polyethylene glycols, to enhance cell-specific targeting using targeting moieties, to aid endosomal escape using fusogenic lipids or pH sensitive polymers, and to improve nuclear entry using nuclear targeting sequences or nuclear localization signal-containing peptides.

Electroporation technologies like nucleofection mediate efficient delivery of DNA and mRNA.

### Exemplary Proteins

Proteins that may be expressed from a gene therapy vector or that may be delivered in a sustained release formulation include VEGF.

The VEGF-A (sometimes referred to as “VEGF-1”) gene contains 8 exons and 7 introns that, by alternative splicing, can form at least six isoforms of the protein. The longest protein isoform is VEGF206, whose mRNA contains the entirety of all eight exons encoding a pre-protein of 232 amino acids, which is processed to the mature form of 206 amino acids. Alternative splicing to produce the different isoforms is focused around exons 6, 7, and 8. The VEGF121 isoform results from joining the splice donor at the end of exon 5 directly to the splice acceptor in exon 8, thereby completely eliminating exons 6 and 7. Exon 6 is especially complex with three different potential splice donors which can ligate to exon 7, resulting in the VEGF206, VEGF189, and VEGF183 isoforms. The 3′ non-transiated end of the gene contains regulatory elements that increase mRNA half-life in response to ischemia.

TABLE 1
Isoforms of human VEGF-A

| | Size (amino | | |
|---|---|---|---|
| Isoform | acids) | Coding exons* | Features |
| VEGF-A₁₂₁ | 121 | 1-5, 8 | Secreted |
| VEGF-A₁₄₅ | 145 | 1-6, 8 | Binds NRP2 but not |
| | | | NRP1; secreted |
| VEGF-A₁₆₅ | 165 | 1-5, 7, 8 | The most abundant |
| | | | and biologically active |
| | | | isoform; secreted; binds |
| | | | NRP1 and NRP2 |
| VEGF-A_{165b} | 165 | 1-5, 7, | Secreted, endogenous |
| | | alternative | inhibitory form of |
| | | exon 8 | VEGF-A165 |
| VEGF-A₁₈₃ | 183 | 1-5, short | Sequestered in ECM |
| | | exon 6, 7, 8 | but released by cleavage |
| VEGF-A₁₈₉ | 189 | 1-8 | Sequestered in ECM but |
| | | | released by cleavage |
| VEGF-A₂₀₆ | 206 | 1-8 plus l | Sequestered in ECM but |
| | | additiona | released by cleavage |
| | | exon 6-encoded | |
| | | sequence | |

*All isoforms contain exons 1-5 and 8, except VEGF-A_{165b}, which contains an alternative exon 8.

The significance of the VEGF isoforms is in their different biological activities. First, the different isoforms have different affinities for the VEGF receptors. At least three VEGF receptors (fltI, flkI/KDR, and neuropilin) are known, which are found in different cell types and at different times during development. FltI mediates cell migration, while KDR is required for the proliferative effects of VEGF (see, e.g., Barleon et al., Blood, 87, 3336-3343 (1996)). While VEGF165 has approximately equal affinity to the flkI/KDR receptor and the fltI receptor, VEGF 121 has a much lower affinity for fltI and binds primarily to KDR (see, e.g., Keyt et al., J. Biol. Chem, 271, 7788-7795 (1996)). Thus, VEGF121 is expected to be biologically inactive in tissues lacking fltI. In the same way, neuropilin is believed to enhance the interaction of VEGF165 with KDR (but not fltI), but has no effect on the binding of VEGF121 to KDR (see, e.g., Gitay-Goren et al., J. Biol. Chem., 271, 5519-5523 (1996), and Park et al., J. Biol. Chem., 269, 25646-25654 (1994)). Second, the different VEGF isoforms differ in their ability to bind heparin and other negatively charged cell matrix components. VEGF121 is missing the basic domains located in exons 6 and 7 which determine interaction with heparin. The presence of heparin can modify both the affinity of the VEGF for its receptors and the residency time in tissue (see, e.g., Keyt et al. (1996), supra, and Cohen et al. (1995), supra). The heparin binding isoforms, such as VEGF165 and VEGF189, will bind extracellular matrix strongly and can be released as biologically active peptides by proteases such as plasmin (see, e.g., Keyt et al. (1996), supra, Athanassiades et al., Bio. Reprod., 59, 643-654 (1998), and Terman et al., Growth Factors, 11, 187-195 (1994)).

The biological significance of the different properties of VEGF isoforms is proven by the phenotype of mice which are unable to make the heparin binding isoform VEGF164/188 (note that the mice VEGF isoforms are one amino acid shorter than the human homologues) (see, e.g., Carmeliet et al., Nat. Med., 5, 495-502 (1999)). Complete deletion of only the VEGF gene is lethal to a mouse embryo even when only one of the two alleles is deleted (see, e.g., Carmeliet et al., Nature, 380, 435-439 (1996)). However, mice can be made with small genomic deletions which encompass exons 6 and 7, thereby making VEGF120 the only isoform that can be produced (see, e.g., Carmeliet et al. (1999), supra). Homozygote mice for VEGF120 are lethal neonatally and suffer from impaired myocardial angiogenesis, which results in decreased contractility and ischemic cardiomyopathy. Thus, the developmental roles of VEGF can be furnished by VEGF120 while the postnatal development of the blood supply, especially to cardiac muscle, depends on the VEGF164/VEGF188 isoforms. This evidence supports the contention that different therapeutic effects might be expected from the production of different isoforms or mixtures of isoforms of VEGF delivered by gene therapy.

Exemplary VEGF sequences include but are not limited to:

VEGF-121: (with Signal Sequence=145 aa)
MNFLLSWVHWSLALLLYLHHAKWSQAAPMAEGGGQNHHEVVKFMDVYQRSYCHPIETLVDIFQEYPDEIEY IFKPSCVPLMRCGGCCNDEGLECVPTEESNITMQIMRIKPHQGQHIGEMSFLQHNKCECRPKKDRARQEKCD KPRR (SEQ ID NO:1), a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:1;

### VEGF145

MNFLLSWVHWSLALLLYLHHAKWSQAAPMAEGGGQNHHEVVKFMDVYQRSYCHPIETLVDIFQEYPDEIEY IFKPSCVPLMRCGGCCNDEGLECVPTEESNITMQIMRIKPHQGQHIGEMSFLQHNKCECRPKKDRARQEKKS VRGKGKGQKRKRKKSRYKSWSVCDKPRR (SEQ ID NO:10)), a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:10;
   VEGF165 (with Signal Sequence=191 aa)
MNFLLSWVHWSLALLLYLHHAKWSQAAPMAEGGGQNHHEVVKFMDVYQRSYCHPIETLVDIFQEYPDEIEY IFKPSCVPLMRCGGCCNDEGLECVPTEESNITMQIMRIKPHQGQHIGEMSFLQHNKCECRPKKDRARQENPC GPCSERRKHLFVQDPQTCKCSCKNTDSRCKARQLELNERTCRCDKPRR (SEQ ID NO:2), a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:2;
   VEGF189 (with Signal Sequence=215 aa)
MNFLLSWVHWSLALLLYLHHAKWSQAAPMAEGGGQNHHEVVKFMDVYQRSYCHPIETLVDIFQEYPDEIEY IFKPSCVPLMRCGGCCNDEGLECVPTEESNITMQIMRIKPHQGQHIGEMSFLQHNKCECRPKKDRARQEKKS VRGKGKGQKRKRKKSRYKSWSVPCGPCSERRKHLFVQDPQTCKCSCKNTDSRCKARQLELNERTCRCDK PRR (SEQ ID NO:3), a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:3;

### VEGF183

MNFLLSWVHWSLALLLYLHHAKWSQAAPMAEGGGQNHHEVVKFMDVYQRSYCHPIETLVDIFQEYPDEIEY IFKPSCVPLMRCGGCCNDEGLECVPTEESNITMQIMRIKPHQGQHIGEMSFLQHNKCECRPKKDRARQEKKS VRGKGKGQKRKRKKSRPCGPCSERRKHLFVQDPQTCKCSCKNTDSRCKARQLELNERTCRCDKPRR(SEQ ID NO:11), a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:11;
or a VEGF encoded by

(SEQ ID NO: 12
ATGAACTTTCTGCTGTCTTGGGTGCATTGGAGCCTTGCCTTGCTGCTCTA
CCTCCACCATGCCAAGTGGTCCCAGGCTGCACCCATGGCAGAAGGAGGAG
GGCAGAATCATCACGAAGTGGTGAAGTTCATGGATGTCTATCAGCGCAGC
TACTGCCATCCAATCGAGACCCTGGTGGACATCTTCCAGGAGTACCCTGA
TGAGATCGAGTACATCTTCAAGCCATCCTGTGTGCCCCTGATGCGATGCG
GGGGCTGCTGCAATGACGAGGGCCTGGAGTGTGTGCCCACTGAGGAGTCC
AACATCACCATGCAGATTATGCGGATCAAACCTCACCAAGGCCAGCACAT
AGGAGAGATGAGCTTCCTACAGCACAACAAATGTGAATGCAGACCAAAGA
AAGATAGAGCAAGACAAGAAAAATGTGACAAGCCGAGGCGGTGA;
(SEQ ID NO: 13)
ATGAACTTTCTGCTGTCTTGGGTGCATTGGAGCCTTGCCTTGCTGCTCTA
CCTCCACCATGCCAAGTGGTCCCAGGCTGCACCCATGGCAGAAGGAGGAG
GGCAGAATCATCACGAAGTGGTGAAGTTCATGGATGTCTATCAGCGCAGC
TACTGCCATCCAATCGAGACCCTGGTGGACATCTTCCAGGAGTACCCTGA
TGAGATCGAGTACATCTTCAAGCCATCCTGTGTGCCCCTGATGCGATGCG
GGGGCTGCTGCAATGACGAGGGCCTGGAGTGTGTGCCCACTGAGGAGTCC
AACATCACCATGCAGATTATGCGGATCAAACCTCACCAAGGCCAGCACAT
AGGAGAGATGAGCTTCCTACAGCACAACAAATGTGAATGCAGACCAAAGA
AAGATAGAGCAAGACAAGAAAAAAAATCAGTTCGAGGAAAGGGAAAGGGG
CAAAAACGAAAGCGCAAGAAATCCCGGTATAAGTCCTGGAGCGTATGTGA
CAAGCCGAGGCGGTGA;
(SEQ ID NO: 14)
ATGAACTTTCTGCTGTCTTGGGTGCATTGGAGCCTTGCCTTGCTGCTCTA
CCTCCACCATGCCAAGTGGTCCCAGGCTGCACCCATGGCAGAAGGAGGAG
GGCAGAATCATCACGAAGTGGTGAAGTTCATGGATGTCTATCAGCGCAGC
TACTGCCATCCAATCGAGACCCTGGTGGACATCTTCCAGGAGTACCCTGA
TGAGATCGAGTACATCTTCAAGCCATCCTGTGTGCCCCTGATGCGATGCG
GGGGCTGCTGCAATGACGAGGGCCTGGAGTGTGTGCCCACTGAGGAGTCC
AACATCACCATGCAGATTATGCGGATCAAACCTCACCAAGGCCAGCACAT
AGGAGAGATGAGCTTCCTACAGCACAACAAATGTGAATGCAGACCAAAGA
AAGATAGAGCAAGACAAGAAAATCCCTGTGGGCCTTGCTCAGAGCGGAGA
AAGCATTTGTTTGTACAAGATCCGCAGACGTGTAAATGTTCCTGCAAAAA
CACAGACTCGCGTTGCAAGGCGAGGCAGCTTGAGTTAAACGAACGTACTT
GCAGATGTGACAAGCCGAGGCGGTGA;
(SEQ ID NO: 15)
ATGAACTTTCTGCTGTCTTGGGTGCATTGGAGCCTTGCCTTGCTGCTCTA
CCTCCACCATGCCAAGTGGTCCCAGGCTGCACCCATGGCAGAAGGAGGAG
GGCAGAATCATCACGAAGTGGTGAAGTTCATGGATGTCTATCAGCGCAGC
TACTGCCATCCAATCGAGACCCTGGTGGACATCTTCCAGGAGTACCCTGA
TGAGATCGAGTACATCTTCAAGCCATCCTGTGTGCCCCTGATGCGATGCG
GGGGCTGCTGCAATGACGAGGGCCTGGAGTGTGTGCCCACTGAGGAGTCC
AACATCACCATGCAGATTATGCGGATCAAACCTCACCAAGGCCAGCACAT
AGGAGAGATGAGCTTCCTACAGCACAACAAATGTGAATGCAGACCAAAGA
AAGATAGAGCAAGACAAGAAAAAAAATCAGTTCGAGGAAAGGGAAAGGGG
CAAAAACGAAAGCGCAAGAAATCCCGTCCCTGTGGGCCTTGCTCAGAGCG
GAGAAAGCATTTGTTTGTACAAGATCCGCAGACGTGTAAATGTTCCTGCA
AAAACACAGACTCGCGTTGCAAGGCGAGGCAGCTTGAGTTAAACGAACGT
ACTTGCAGATGTGACAAGCCGAGGCGGTGA;
(SEQ ID NO: 16)
ATGAACTTTCTGCTGTCTTGGGTGCATTGGAGCCTTGCCTTGCTGCTCTA
CCTCCACCATGCCAAGTGGTCCCAGGCTGCACCCATGGCAGAAGGAGGAG
GGCAGAATCATCACGAAGTGGTGAAGTTCATGGATGTCTATCAGCGCAGC
TACTGCCATCCAATCGAGACCCTGGTGGACATCTTCCAGGAGTACCCTGA
TGAGATCGAGTACATCTTCAAGCCATCCTGTGTGCCCCTGATGCGATGCG
GGGGCTGCTGCAATGACGAGGGCCTGGAGTGTGTGCCCACTGAGGAGTCC
AACATCACCATGCAGATTATGCGGATCAAACCTCACCAAGGCCAGCACAT
AGGAGAGATGAGCTTCCTACAGCACAACAAATGTGAATGCAGACCAAAGA
AAGATAGAGCAAGACAAGAAAAAAAATCAGTTCGAGGAAAGGGAAAGGGG
CAAAAACGAAAGCGCAAGAAATCCCGGTATAAGTCCTGGAGCGTTCCCTG
TGGGCCTTGCTCAGAGCGGAGAAAGCATTTGTTTGTACAAGATCCGCAGA
CGTGTAAATGTTCCTGCAAAAACACAGACTCGCGTTGCAAGGCGAGGCAG
CTTGAGTTAAACGAACGTACTTGCAGATGTGACAAGCCGAGGCGGTGA;
or
(SEQ ID NO: 17)
ATGAACTTTCTGCTGTCTTGGGTGCATTGGAGCCTTGCCTTGCTGCTCTA
CCTCCACCATGCCAAGTGGTCCCAGGCTGCACCCATGGCAGAAGGAGGAG
GGCAGAATCATCACGAAGTGGTGAAGTTCATGGATGTCTATCAGCGCAGC
TACTGCCATCCAATCGAGACCCTGGTGGACATCTTCCAGGAGTACCCTGA
TGAGATCGAGTACATCTTCAAGCCATCCTGTGTGCCCCTGATGCGATGCG
GGGGCTGCTGCAATGACGAGGGCCTGGAGTGTGTGCCCACTGAGGAGTCC
AACATCACCATGCAGATTATGCGGATCAAACCTCACCAAGGCCAGCACAT
AGGAGAGATGAGCTTCCTACAGCACAACAAATGTGAATGCAGACCAAAGA
AAGATAGAGCAAGACAAGAAAAAAAATCAGTTCGAGGAAAGGGAAAGGGG
CAAAAACGAAAGCGCAAGAAATCCCGGTATAAGTCCTGGAGCGTGTACGT
TGGTGCCCGCTGCTGTCTAATGCCCTGGAGCCTCCCTGGCCCCCATCCCT
GTGGGCCTTGCTCAGAGCGGAGAAAGCATTTGTTTGTACAAGATCCGCAG
ACGTGTAAATGTTCCTGCAAAAACACAGACTCGCGTTGCAAGGCGAGGCA
GCTTGAGTTAAACGAACGTACTTGCAGATGTGACAAGCCGAGGCGGTGA.

Other exemplary proteins for use in the vectors or in sustained release formulations include but are not limited to: VEGFC, VEGFD, ANG1, ANG2, PDGFA, PDGFB, PLGF, TPO, HGF, FGF1, FGF2, TGFB, TNFA, CXCL2, CXCL8, CCL2, IL6, IL8, IL22, or EDN1.

An exemplary VEGFC has

MHLLGFFSVA CSLLAAALLP GPREAPAAAA AFESGLDLSD
AEPDAGEATA YASKDLEEQL RSVSSVDELM TVLYPEYWKM
YKCQLRKGGW QHNREQANLN SRTEETIKFA AAHYNTEILK
SIDNEWRKTQ CMPREVCIDV GKEFGVATNT FFKPPCVSVY
RCGGCCNSEG LQCMNTSTSY LSKTLFEITV PLSQGPKPVT
ISFANHTSCR CMSKLDVYRQ VHSIIRRSLP ATLPQCQAAN
KTCPTNYMWN NHICRCLAQE DFMFSSDAGD DSTDGFHDIC
GPNKELDEET CQCVCRAGLR PASCGPHKEL DRNSCQCVCK
NKLFPSQCGA NREFDENTCQ CVCKRTCPRN QPLNPGKCAC
ECTESPQKCL LKGKKFHHQT CSCYRRPCTN ROKACEPGFS
YSEEVCRCVP SYWKRPQMS

(SEQ ID NO:21), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:21.

An exemplary VEGFD has

MYREWVVVNV FMMLYVQLVQ GSSNEHGPVK RSSQSTLERS
EQQIRAASSL EELLRITHSE DWKLWRCRLR LKSFTSMDSR
SASHRSTRFA ATFYDIETLK VIDEEWQRTQ CSPRETCVEV
ASELGKSTNT FFKPPCVNVF RCGGCCNEES LICMNTSTSY
ISKQLFEISV PLTSVPELVP VKVANHTGCK CLPTAPRHPY
SIIRRSIQIP EEDRCSHSKK LCPIDMLWDS NKCKCVLQEE
NPLAGTEDHS HLQEPALCGP HMMFDEDRCE CVCKTPCPKD
LIQHPKNCSC FECKESLETC CQKHKLFHPD TCSCEDRCPF
HTRPCASGKT ACAKHCRFPK EKRAAQGPHS RKNP

(SEQ ID NO:22), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:22.

An exemplary ANG1 has

MTVFLSFAFL AAILTHIGCS NQRRSPENSG RRYNRIQHGQ
CAYTFILPEH DGNCRESTTD QYNTNALQRD APHVEPDFSS
QKLQHLEHVM ENYTQWLQKL ENYIVENMKS EMAQIQQNAV
QNHTATMLEI GTSLLSQTAE QTRKLTDVET QVLNQTSRLE
IQLLENSLST YKLEKQLLQQ TNEILKIHEK NSLLEHKILE
MEGKHKEELD TLKEEKENLQ GLVTRQTYII QELEKQLNRA
TTNNSVLQKQ QLELMDTVHN LVNLCTKEGV LLKGGKREEE
KPFRDCADVY QAGFNKSGIY TIYINNMPEP KKVFCNMDVN
GGGWTVIQHR EDGSLDFQRG WKEYKMGFGN PSGEYWLGNE
FIFAITSQRQ YMLRIELMDW EGNRAYSQYD RFHIGNEKQN
YRLYLKGHTG TAGKQSSLIL HGADFSTKDA DNDNCMCKCA
LMLTGGWWFD ACGPSNLNGM FYTAGQNHGK LNGIKWHYFK
GPSYSLRSTT MMIRPLDF

(SEQ ID NO:23), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:23.

An exemplary ANG2 has

MWQIVFFTLS CDLVLAAAYN NFRKSMDSIG KKQYQVQHGS
CSYTFLLPEM DNCRSSSSPY VSNAVQRDAP LEYDDSVQRL
QVLENIMENN TQWLMKLENY IQDNMKKEMV EIQQNAVQNQ
TAVMIEIGTN LLNQTAEQTR KLTDVEAQVL NQTTRLELQL
LEHSLSTNKL EKQILDQTSE INKLQDKNSF LEKKVLAMED
KHIIQLQSIK EEKDQLQVLV SKQNSIIEEL EKKIVTATVN
NSVLQKQQHD LMETVNNLLT MMSTSNSAKD PTVAKEEQIS
FRDCAEVFKS GHTTNGIYTL TFPNSTEEIK AYCDMEAGGG
GWTIIQRRED GSVDFQRTWK EYKVGFGNPS GEYWLGNEFV
SQLTNQQRYV LKIHLKDWEG NEAYSLYEHF YLSSEELNYR
IHLKGLTGTA GKISSISQPG NDFSTKDGDN DKCICKCSQM
LTGGWWFDAC GPSNLNGMYY PQRQNTNKFN GIKWYYWKGS
GYSLKATTMM IRPADF

(SEQ ID NO:24), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:24.

An exemplary PDGFA has

MRTLACLLLL GCGYLAHVLA EEAEIPREVI ERLARSQIHS IRDLQRLLEI DSVGSEDSLD
TSLRAHGVHA TKHVPEKRPL PIRRKRSIEE AVPAVCKTRT VIYEIPRSQV DPTSANFLIW
PPCVEVKRCT GCCNTSSVKC QPSRVHHRSV KVAKVEYVRK KPKLKEVQVR LEEHLECACA
TTSLNPDYRE EDTGRPRESG KKRKRKRLKP T

(SEQ ID NO:25), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:25.

An exemplary PDGFB has

MNRCWALFLS LCCYLRLVSA EGDPIPEELY EMLSDHSIRS FDDLQRLLHG DPGEEDGAEL
DLNMTRSHSG GELESLARGR RSLGSLTIAE PAMIAECKTR TEVFEISRRL IDRTNANFLV
WPPCVEVQRC SGCCNNRNVQ CRPTQVQLRP VQVRKIEIVR KKPIFKKATV TLEDHLACKC
ETVAAARPVT RSPGGSQEQR AKTPQTRVTI RTVRVRRPPK GKHRKFKHTH DKTALKETLG
A

(SEQ ID NO:26), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:26.

An exemplary PLGF has

MPVMRLFPCF LQLLAGLALP AVPPQQWALS AGNGSSEVEV VPFQEVWGRS YCRALERLVD
VVSEYPSEVE HMFSPSCVSL LRCTGCCGDE NLHCVPVETA NVTMQLLKIR SGDRPSYVEL
TFSQHVRCEC RPLREKMKPE RRRPKGRGKR RREKQRPTDC HLCGDAVPRR

(SEQ ID NO:27), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:27

An exemplary TPO has

MELTELLLVV MLLPTARLTL SSPAPPACDL RVLSKLLRDS HVLHSKLSQC PEVHPLPTPV
LLPAVDFSLG EWKTQMEETK AQDILGAVTL LLEGVMAARG QLGPTCLSSL LGQLSEQVRL
LLGALQSLLG TQLPPQGRTT AHKDPNAIFL SFQHLLRGKV RFLMLVGGST LCVRRAPPTT
AVPSRTSLVL TLNELPNRTS GLLETNFTAS ARTTGSGLLK WQQGFRAKIP GLLNQTSRSL
DQIPGYLNRI HELLNGTRGL FPGPSRRTLG APDISSETSD TGSLPPNLQP GYSPSPTHPP
TGQYTLFPLP PTLPTPVVQL HPLLPDPSAP TPTPTSPLLN TSYTHCQNLS QEG

(SEQ ID NO:28), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:28.

An exemplary HGF has

MWVTKLLPAL LLQHVLLHLL LLPIAIPYAE GQRKRRNTIH EFKKSAKTTL IKIDPALKIK
TKKVNTADQC ANRCTRNKGL PFTCKAFVFD KARKQCLWFP FNSMSSGVKK EFGHEFDLYE
NKDYIRNCII GKGRSYKGTV SITKSGIKCQ PWSSMIPHEH SFLPSSYRGK DLQENYCRNP
RGEEGGPWCF TSNPEVRYEV CDIPQCSEVE CMTQNGESYR GLMDHTESGK ICQRWDHQTP
HRHKFLPERY PDKGFDDNYC RNPDGQPRPW CYTLDPHTRW EYCAIKTCAD NTMNDTDVPL
ETTECIQGQG EGYRGTVNTI WNGIPCQRWD SQYPHEHDMT PENFKCKDLR ENYCRNPDGS
ESPWCFTTDP NIRVGYCSQI PNCDMSHGQD CYRGNGKNYM GNLSQTRSGL TCSMWDKNME
DLHRHIFWEP DASKLNENYC RNPDDDAHGP WCYTGNPLIP WDYCPISRCE GDTTPTIVNL
DHPVISCAKT KQLRVVNGIP TRTNIGWMVS LRYRNKHICG GSLIKESWVL TARQCFPSRD
LKDYEAWLGI HDVHGRGDEK CKQVLNVSQL VYGPEGSDLV LMKLARPAVL DDFVSTIDLP
NYGCTIPEKT SCSVYGWGYT GLINYDGLLR VAHLYIMGNE KCSQHHRGKV TLNESEICAG
AEKIGSGPCE GDYGGPLVCE QHKMRMVLGV IVPGRGCAIP NRPGIFVRVA YYAKWIHKII
LTYKVPQS

(SEQ ID NO:29), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:29.

An exemplary FGF1 has

MWVTKLLPAL LLQHVLLHLL LLPIAIPYAE GQRKRRNTIH EFKKSAKTTL IKIDPALKIK
TKKVNTADQC ANRCTRNKGL PFTCKAFVFD KARKQCLWFP FNSMSSGVKK EFGHEFDLYE
NKDYIRNCII GKGRSYKGTV SITKSGIKCQ PWSSMIPHEH SFLPSSYRGK DLQENYCRNP
RGEEGGPWCF TSNPEVRYEV CDIPQCSEVE CMTCNGESYR GLMDHTESGK ICQRWDHQTP
HRHKFLPERY PDKGFDDNYC RNPDGQPRPW CYTLDPHTRW EYCAIKTCAD NTMNDTDVPL
ETTECIQGQG EGYRGTVNTI WNGIPCQRWD SQYPHEHDMT PENFKCKDLR ENYCRNPDGS
ESPWCFTTDP NIRVGYCSQI PNCDMSHGQD CYRGNGKNYM GNLSQTRSGL TCSMWDKNME
DLHRHIFWEP DASKLNENYC RNPDDDAHGP WCYTGNPLIP WDYCPISRCE GDTTPTIVNL
DHPVISCAKT KQLRVVNGIP TRTNIGWMVS LRYRNKHICG GSLIKESWVL TARQCFPSRD
LKDYEAWLGI HDVHGRGDEK CKQVLNVSQL VYGPEGSDLV LMKLARPAVL DDFVSTIDLP
NYGCTIPEKT SCSVYGWGYT GLINYDGLLR VAHLYIMGNE KCSQHHRGKV TLNESEICAG
AEKIGSGPCE GDYGGPLVCE QHKMRMVLGV IVPGRGCAIP NRPGIFVRVA YYAKWIHKII
LTYKVPQS

(SEQ ID NO:30), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:30.

An exemplary FGF2 has

MVGVGGGDVE DVTPRPGGCQ ISGRGARGCN GIPGAAAWEA ALPRRRPRRH PSVNPRSRAA
GSPRTRGRRT EERPSGSRLG DRGRGRALPG GRLGGRGRGR APERVGGRGR GRGTAAPRAA
PAARGSRPGP AGTMAAGSIT TLPALPEDGG SGAFPPGHFK DPKRLYCKNG GFFLRIHPDG
RVDGVREKSD PHIKLQLQAE ERGVVSIKGV CANRYLAMKE DGRLLASKCV TDECFFFERL
ESNNYNTYRS RKYTSWYVAL KRTGQYKLGS KTGPGQKAIL FLPMSAKS

(SEQ ID NO:31), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:31.

An exemplary TGFB has

MPPSGLRLLP LLLPLLWLLV LTPGRPAAGL STCKTIDMEL VKRKRIEAIR GQILSKLRLA
SPPSQGEVPP GPLPEAVLAL YNSTRDRVAG ESAEPEPEPE ADYYAKEVTR VLMVETHNEI
YDKFKQSTHS IYMFFNTSEL REAVPEPVLL SRAELRLLRL KLKVEQHVEL YQKYSNNSWR
YLSNRLLAPS DSPEWLSFDV TGVVRQWLSR GGEIEGFRLS AHCSCDSRDN TLQVDINGFT
TGRRGDLATI HGMNRPFLLL MATPLERAQH LQSSRHRRAL DTNYCFSSTE KNCCVRQLYI
DFRKDLGWKW IHEPKGYHAN FCLGPCPYIW SLDTQYSKVL ALYNQHNPGA SAAPCCVPQA
LEPLPIVYYV GRKPKVEQLS NMIVRSCKCS

(SEQ ID NO:32), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:32.

An exemplary TNFA has

MSTESMIRDV ELAEEALPKK TGGPQGSRRC LFLSLFSFLI VAGATTLFCL LHFGVIGPQR
EEFPRDLSLI SPLAQAVRSS SRTPSDKPVA HVVANPQAEG QLQWLNRRAN ALLANGVELR
DNQLVVPSEG LYLIYSQVLF KGQGCPSTHV LLTHTISRIA VSYQTKVNLL SAIKSPCQRE
TPEGAEAKPW YEPIYLGGVF QLEKGDRLSA EINRPDYLDF AESGQVYFGI IAL

(SEQ ID NO:33), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:33.

An exemplary CXCL2 has

MARATLSAAP SNPRLLRVAL LLLLLVAASR RAAGAPLATE LRCQCLQTLQ GIHLKNIQSV
KVKSPGPHCA QTEVIATLKN GQKACLNPAS PMVKKIIEKM LKNGKSN

(SEQ ID NO:34), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:34.

An exemplary CXCL8 has

MTSKLAVALL AAFLISAALC EGAVLPRSAK ELRCQCIKTY SKPFHPKFIK ELRVIESGPH
CANTEIIVKL SDGRELCLDP KENWVQRVVE KFLKRAENS

(SEQ ID NO:35), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:35.

An exemplary CCL2 has

MKVSAALLCL LLIAATFIPQ GLAQPDAINA PVTCCYNFTN RKISVQRLAS YRRITSSKCP
KEAVIFKTIV AKEICADPKQ KWVQDSMDHL DKQTQTPKT

(SEQ ID NO:36), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:36.

An exemplary IL6 has

MNSFSTSAFG PVAFSLGLLL VLPAAFPAPV PPGEDSKDVA APHRQPLTSS ERIDKQIRYI
LDGISALRKE TQNKSNMCES SKEALAENNL NLPKMAEKDG CFQSGFNEET CLVKIITGLL
EFEVYLEYLQ NRFESSEEQA RAVQMSTKVL IQFLQKKAKN LDAITTPDPT TNASLLTKLQ
AQNQWLQDMT THLILRSFKE FLQSSLRALR QM

(SEQ ID NO:37), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:37.

An exemplary IL22 has

MAALQKSVSS FLMGTLATSC LLLLALLVQG GAAAPISSHC RLDKSNFQQP YITNRTFMLA
KEASLADNNT DVRLIGEKLF HGVSMSERCY LMKQVLNFTL EEVLFPQSDR FQPYMQEVVP
FLARLSNRLS TCHIEGDDLH IQRNVQKLKD TVKKLGESGE IKAIGELDLL FMSLRNACI

(SEQ ID NO:39), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:39.

An exemplary EDN1 has

MDYLLMIFSL LFVACQGAPE TAVLGAELSA VGENGGEKPT PSPPWRLRRS KRCSCSSLMD
KECVYFCHLD IIWVNTPEHV VPYGLGSPRS KRALENLLPT KATDRENRCQ CASQKDKKCW
NFCQAGKELR AEDIMEKDWN NHKKGKDCSK LGKKCIYQQL VRGRKIRRSS EEHLRQTRSE
TMRNSVKSSF HDPKLKGKPS RERYVTHNRA HW

(SEQ ID NO:38), or is an isoform thereof, a polypeptide with at least 80%, 82%, 84%, 85%, 86%, 87%, 89%, 90%, 92%, 94%, 95%, 96%, 97%, 98% or 99% amino acid sequence identity thereto, or a portion thereof with the activity of SEQ ID NO:38.

VEGFA, VEGFC, VEGFD, ANG1, ANG2, PDGFA, PDGFB, PLGF, TPO, HGF, FGF1, FGF2, TGFB, TNFA, CXCL2, CXCL8, CCL2, IL6, IL8, IL22, or EDN1 encoding nucleic acid molecules can be inserted into an expression construct. A nucleic acid molecule can be cloned into any suitable expression construct and can be used to transform or transfect any suitable host cell, e.g., adipocytes or fat stem cells. The selection of expression and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987)).

Variants of the sequences disclosed above are envisioned. For example, a variant may include one or more conservative amino acid substitutions—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

The disclosure also envisions a peptide, polypeptide or fusion polypeptide with non-conservative substitutions. Non-conservative substitutions entail exchanging a member of one of the classes described above for another.

Acid addition salts of the peptide, polypeptide or fusion polypeptide or of amino residues of the peptide, polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

Suitable expression constructs include those designed for propagation and expansion or for expression or both. Examples of suitable expression constructs include plasmids, phagemids, cosmids, viruses, and other vehicles derived from viral or bacterial sources. Any of these expression constructs can be manipulated to include a nucleic acid sequence and can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Plasmids are genetically engineered circular double-stranded DNA molecules and can be designed to contain an expression cassette comprising a nucleic acid molecule encoding VEGF. By complexing the plasmid with liposomes, the efficiency of gene transfer in general is improved. While the liposomes used for plasmid-mediated gene transfer strategies have various compositions, they are typically synthetic cationic lipids. Advantages of plasmid-liposome complexes include their ability to transfer large nucleic acid sequences and their relatively low immunogenicity. While plasmids are suitable for use in the disclosure, the expression construct may be a viral vector.

### Exemplary Adenovirus Vectors

Adenovirus is a 36 kb double-stranded DNA virus that efficiently transfers DNA in vivo to a variety of different target cell types. The Ad vector can be produced in high titers and can efficiently transfer DNA to replicating and non-replicating cells. The Ad vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Given that the human adenovirus serotype 5 (Ad5) genome has been completely sequenced, the adenoviral vector is described herein with respect to the Ad5 serotype. The Ad vector can be any adenoviral vector capable of growth in a cell, which is in some significant part (although not necessarily substantially) derived from or based upon the genome of an adenovirus. The Ad vector can be based on the genome of any suitable wild-type adenovirus. The Ad vector may be derived from the genome of a wild-type adenovirus of group C, especially of serotype 2 or 5. Ad vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

The Ad vector may be replication-deficient. By “replication-deficient” is meant that the Ad vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient Ad vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). The replication-deficient Ad vector may comprise an adenoviral genome deficient in two or more gene functions required for viral replication. The two or more regions of the adenoviral genome may be selected from the group consisting of the E1, E2, and E4 regions. The replication-deficient adenoviral vector may comprise a deficiency in at least one replication-essential gene function of the E1 region (denoted an E1⁻ deficient adenoviral vector). The E1 region of the adenoviral genome comprises the E1A region and the E1B region. The E1A and E1B regions comprise nucleic acid sequences coding for multiple peptides by virtue of RNA splicing. A deficiency of a gene function encoded by either or both of the E1A and/or E1B regions of the adenoviral genome (e.g., a peptide that performs a function required for replication) is considered a deficiency of a gene function of the E1 region in the context of the disclosure. In addition to such a deficiency in the E1 region, the recombinant adenovirus also can have a mutation in the major late promoter (MLP), as discussed in International Patent Application WO 00/00628. The vector may be deficient in at least one replication-essential gene function of the E1 region and at least part of the nonessential E3 region (e.g., an Xba I deletion of the E3 region) (denoted an E1/E3-deficient adenoviral vector).

The adenoviral vector may be “multiply deficient,” meaning that the adenoviral vector is deficient in one or more gene functions required for viral replication in each of two or more regions of the adenoviral genome. For example, the aforementioned E1-deficient or E1/E3-deficient Ad vector can be further deficient in at least one replication-essential gene function of the E4 region (denoted an E1/E4-deficient adenoviral vector). An adenoviral vector deleted of the entire E4 region can elicit a lower host immune response.

Alternatively, the Ad vector lacks replication-essential gene functions in all or part of the E1 region and all or part of the E2 region (denoted an E1/E2-deficient adenoviral vector). Ad vectors lacking replication-essential gene functions in all or part of the E1 region, all or part of the E2 region, and all or part of the E3 region also are contemplated herein. If the Ad vector is deficient in a replication-essential gene function of the E2A region, the vector in one embodiment does not comprise a complete deletion of the E2A region, which is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. The multiply replication-deficient adenoviral vector may contain this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5.

The Ad vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it may be preferred that at least either the viral (i.e., adenoviral) inverted terminal repeats (Ad ITRs) and one or more promoters or the Ad ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the Ad ITRs and one or more promoters intact, the exogenous insert capacity of the adenovirus is approximately 35 kb. Alternatively, a multiply deficient Ad vector that contains only an Ad ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts. Suitable replication-deficient Ad vectors, including multiply deficient Ad vectors, are disclosed in U.S. Pat. Nos. 5,851,806 and 5,994,106 and International Patent Applications WO 95/34671 and WO 97/21826. An adenoviral vector for use in the methods is that described in International Patent Application WO 02/00906.

It should be appreciated that the deletion of different regions of the Ad vector can alter the immune response of a mammal exposed to the Ad vector. In particular, the deletion of different regions can reduce the inflammatory response generated by the Ad vector. Furthermore, the Ad vector's coat protein can be modified so as to decrease the Ad vector's ability or inability to be recognized by a neutralizing antibody directed against the wild-type coat protein, as described in International Patent Application WO 98/40509.

The adenoviral vector, when multiply replication-deficient, especially in replication-essential gene functions of the E1 and E4 regions, e.g., includes a spacer element to provide viral growth in a complementing cell line similar to that achieved by singly replication deficient Ad vectors, particularly an Ad vector comprising a deficiency in the E4 region. A spacer sequence is defined in the disclosure as any sequence of sufficient length to restore the size of the adenoviral genome to approximately the size of a wild-type adenoviral genome, such that the Ad vector is efficiently packaged into viral particles. The spacer element can contain any sequence or sequences which are of the desired length. The spacer element sequence can be coding or non-coding and native or non-native with respect to the adenoviral genome, but does not restore the replication-essential function to the deficient region. The spacer can be of any suitable size, desirably at least about 15 base pairs (e.g., between about 15 base pairs and about 12,000 base pairs), about 100 base pairs to about 10,000 base pairs, about 500 base pairs to about 8,000 base pairs, about 1,500 base pairs to about 6,000 base pairs, or about 2,000 to about 3,000 base pairs. The size of the spacer is limited only by the size of the insert that the Ad vector will accommodate (e.g., approximately 38 kb). In the absence of a spacer, production of fiber protein and/or viral growth of the multiply replication-deficient Ad vector is reduced by comparison to that of a singly replication-deficient Ad vector. However, inclusion of the spacer in at least one of the deficient adenoviral regions, e.g., the E4 region, can counteract this decrease in fiber protein production and viral growth. The use of a spacer in an Ad vector is described in U.S. Pat. No. 5,851,806.

The Ad vector may contain a packaging domain. The packaging domain can be located at any position in the adenoviral genome, so long as the adenoviral genome is packaged into adenoviral particles. The packaging domain may be located downstream of the E1 region. The packaging domain may be located downstream of the E4 region. The replication-deficient Ad vector may lacks all or part of the E1 region and the E4 region. A spacer may be inserted into the E4 region, a desired exogenous nucleic acid sequence of interest (e.g., a nucleic acid sequence encoding TNF-α) is located in the E1 region, and the packaging domain is located downstream of the E4 region. By relocating the packaging domain, the amount of potential overlap between the Ad vector and the cellular/helper virus genome used to propagate the Ad vector is reduced so as to reduce the probability of obtaining a replication-competent Ad vector.

The coat proteins of the Ad vector can be manipulated to alter the binding specificity of the resulting adenoviral particle. Suitable modifications to the coat proteins include, but are not limited to, insertions, deletions, or replacements in the adenoviral fiber, penton, pIX, pIIIa, pVI, or hexon proteins, or any suitable combination thereof, including insertions of various native or non-native ligands into portions of such coat proteins. Examples of Ad vectors with modified binding specificity are described in, e.g., U.S. Pat. Nos. 5,871,727, 5,885,808, and 5,922,315. Modified Ad vector particles include those described in, for example, Wickham et al., J. Virol., 71(10), 7663-9 (1997), Cripe et al., Cancer Res., 61(7), 2953-60 (2001), van Deutekom et al., J. Gene Med., 1(6), 393-9 (1999), McDonald et al., J. Gene Med., 1(2), 103-10 (1999), Staba et al., Cancer Gene Ther., 7(1), 13-9 (2000), Wickham, Gene Ther., 7(2), 110-4 (2000), Kibbe et al., Arch. Surg., 135(2), 191-7 (2000), Harari et al., Gene Ther., 6(5), 801-7 (2000), Bouri et al., Hum Gene Ther., 10(10), 1633-40 (1999), Wickham et al., Nat. Biotechnol., 14(11), 1570-3 (1996), Wickham et al., Cancer Immunol. Immunother., 45(3-4), 149-51 (1997), and Wickham et al., Gene Ther., 2(10), 750-6 (1995), and U.S. Pat. Nos. 5,559,099; 5,712,136; 5,731,190; 5,770,442; 5,801,030; 5,846,782; 5,962,311; 5,965,541; 6,057,155; 6,127,525; and 6,153,435; and International Patent Applications WO 96/07734, WO 96/26281, WO 97/20051, WO 98/07865, WO 98/07877, WO 98/40509, WO 98/54346, WO 00/15823, and WO 01/58940.

Replication-deficient Ad vectors are typically produced in complementing cell lines that provide gene functions not present in the replication-deficient Ad vectors, but required for viral propagation, at appropriate levels in order to generate high titers of viral vector stock. A cell line complements for at least one and optionally all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons, which comprise minimal adenoviral sequences, such as only Ad ITRs and the packaging signal or only Ad ITRs and an adenoviral promoter). The complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line may be further characterized in that it contains the complementing genes in a non-overlapping fashion with the adenoviral vector, which minimizes, and practically eliminates, the possibility of the vector genome recombining with the cellular DNA. Accordingly, the presence of replication-competent adenoviruses (RCA) is minimized if not avoided in the vector stock, which, therefore, is suitable for certain therapeutic purposes, especially gene therapy purposes. The lack of RCA in the vector stock avoids the replication of the Ad vector in non-complementing cells. The construction of complementing cell lines involves standard molecular biology and cell culture techniques, such as those described by Sambrook et al. (1989), supra, and Ausubel et al. (1984), supra. Complementing cell lines for producing adenoviral vectors include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)).

### Expression Cassettes for Nucleic Acid Based Delivery

The selection of an expression construct for use in the disclosure may depend on a variety of factors such as, for example, the host, immunogenicity of the expression construct, the desired duration of protein production, the target cell, and the like. As each type of expression construct has distinct properties, a researcher has the freedom to tailor the disclosure to any particular situation. Moreover, more than one type of expression construct can be used, if desired.

Accordingly, the nucleic acid molecule encoding, for example, VEGF is operably linked to regulatory sequences necessary for expression, especially a promoter. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the disclosure to provide for transcription of a particular nucleic acid sequence. The promoter may be capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

The vector may employ a viral promoter. Suitable viral promoters are known in the art and include, for instance, cytomegalovirus (CMV) promoters, such as the CMV immediate-early promoter, promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., PNAS, 78, 144-145 (1981)), promoters derived from SV40 or Epstein Barr virus, an adeno-associated viral promoter, such as the p5 promoter, and the like. The viral promoter may be an adenoviral promoter, such as the Ad2 or Ad5 major late promoter and tripartite leader, a CMV promoter, or an RSV promoter.

Many of the above-described promoters are constitutive promoters. Instead of being a constitutive promoter, the promoter can be an inducible promoter, i.e., a promoter that is up- and/or down-regulated in response to appropriate signals. Examples of suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed. The promoter sequence that regulates expression of the nucleic acid sequence can contain at least one heterologous regulatory sequence responsive to regulation by an exogenous agent. The regulatory sequences may be responsive to exogenous agents such as, but not limited to, drugs, hormones, or other gene products. For example, the regulatory sequences, e.g., promoter, may be responsive to glucocorticoid receptor-hormone complexes, which, in turn, enhance the level of transcription of a therapeutic peptide or a therapeutic fragment thereof.

One of ordinary skill in the art will appreciate that each promoter drives transcription, and, therefore, protein expression, differently with respect to the time and amount of protein produced. For example, the CMV promoter is characterized as having peak activity shortly after transduction, i.e., about 24 hours after transduction, then quickly tapering off. On the other hand, the RSV promoter's activity increases gradually, reaching peak activity several days after transduction, and maintains a high level of activity for several weeks. Indeed, sustained expression driven by an RSV promoter has been observed in all cell types studied, including, for instance, liver cells, lung cells, spleen cells, diaphragm cells, skeletal muscle cells, and cardiac muscle cells. Thus, a promoter can be selected for use in the disclosure by matching its particular pattern of activity with the desired pattern and level of expression of a nucleic acid sequence of interest. Alternatively, a hybrid promoter can be constructed which combines the desirable aspects of multiple promoters. For example, a CMV-RSV hybrid promoter combining the CMV promoter's initial rush of activity with the RSV promoter's high maintenance level of activity may be employed. It is also possible to select a promoter with an expression profile that can be manipulated by an investigator.

With respect to promoters, nucleic acid sequences, selectable markers, and the like, located on an expression construct, such elements can be present as part of a cassette, either independently or coupled. In the context of the disclosure, a “cassette” is a particular base sequence that possesses functions, which facilitate subcloning, and recovery of nucleic acid sequences (e.g., one or more restriction sites) or expression (e.g., polyadenylation or splice sites) of particular nucleic acid sequences.

Construction of a nucleic acid sequence operably linked to regulatory sequences necessary for expression is well within the skill of the art (see, for example, Sambrook et al. (1989), supra). With respect to the expression of nucleic acid sequences according to the disclosure, the ordinary skilled artisan is aware that different genetic signals and processing events control levels of nucleic acids and proteins/peptides in a cell, such as, for instance, transcription, mRNA translation, and post-transcriptional processing. Transcription of DNA into RNA requires a functional promoter, as described herein.

Protein expression is dependent on the level of RNA transcription that is regulated by DNA signals, and the levels of DNA template. Similarly, translation of mRNA requires, at the very least, an AUG initiation codon, which is usually located within 10 to 100 nucleotides of the 5′ end of the message. Sequences flanking the AUG initiator codon have been shown to influence its recognition by eukaryotic ribosomes, with conformity to a perfect Kozak consensus sequence resulting in optimal translation (see, e.g., Kozak, J. Mol. Biol., 196, 947-950 (1987)). Also, successful expression of an exogenous nucleic acid in a cell can require post-translational modification of a resultant protein. Thus, production of a protein can be affected by the efficiency with which DNA (or RNA) is transcribed into mRNA, the efficiency with which mRNA is translated into protein, and the ability of the cell to carry out post-translational modification. These are all factors of which the ordinary skilled artisan is aware and is capable of manipulating using standard means to achieve the desired end result.

Along these lines, to optimize protein production, the nucleic acid molecule may further comprise a polyadenylation site following the coding region of the nucleic acid sequence. Also, the proper transcription signals (and translation signals, where appropriate) may be correctly arranged such that the nucleic acid sequence will be properly expressed in the cells into which it is introduced. Moreover, if the nucleic acid sequence encodes a protein or peptide, which is a processed or secreted protein or acts intracellularly, e.g., the nucleic acid sequence further comprises the appropriate sequences for processing, secretion, intracellular localization, and the like.

It will be appreciated that the expression construct can comprise multiple nucleic acid molecules. For example, the expression construct can comprise multiple copies of a nucleic acid molecule, each copy operably linked to a different promoter or to identical promoters. Moreover, any nucleic acid molecule described herein can be altered from its native form to increase or decrease a desired effect (e.g., to increase its therapeutic effect). For example, a cytoplasmic form of a nucleic acid molecule can be converted to a secreted form by incorporating a signal peptide into the encoded gene product.

### Delivery Vehicles

Delivery vehicles include, for example, viral vectors, microparticles, nanoparticles, liposomes and other lipid-containing complexes, and other macromolecular complexes capable of mediating delivery of a gene or protein to a host cell, e.g., to provide for recombinant expression of a polypeptide encoded by the gene. Vectors or vehicles can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors or vehicles within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors, which are present in cells such as adipocytes, or proteins, which are present in for example, nanoparticles or microparticles including liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene viral vectors are described below. Cells or vehicles may be administered via any route including local administration, e.g., topical, subdermal, or subcutaneous administration.

### Exemplary Formulations and Dosages

The polypeptides or portions thereof, or cells having nucleic acid encoding a polypeptide or portion thereof, can be formulated as pharmaceutical compositions and administered to a mammalian host, such as a human patient in a variety of forms adapted to the chosen route of administration, e.g., parenterally, or by intravenous, intramuscular, topical or subcutaneous routes.

In one embodiment, a sustained release formulation comprising the polypeptides or portions thereof and fat cells, or cells having nucleic acid encoding the polypeptide or portion thereof, may be administered by infusion or injection. Solutions of the polypeptides or fusions thereof, or nucleic acid encoding the polypeptide or portion thereof, or salts thereof, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The amount of the polypeptides or portions thereof, or cells having the nucleic acid encoding the polypeptide or portion therein, required for use alone or with other agents will vary with the route of administration, the nature of the condition being treated and the age and condition of the patient and will be ultimately at the discretion of the attendant physician or clinician.

The polypeptides or portions thereof, or cells having nucleic acid encoding the polypeptide or portion thereof, may be conveniently administered in unit dosage form; for example, containing 5 to 1000 mg, conveniently 10 to 750 mg, or conveniently 50 to 500 mg of active ingredient per unit dosage form.

A suitable dose may be in the range of from about 0.5 to about 100 mg/kg, e.g., from about 10 to about 75 mg/kg of body weight, such as 3 to about 50 mg per kilogram body weight, for example in the range of 6 to 90 mg/kg, e.g., in the range of 15 to 60 mg/kg.

### Exemplary Particle Formulations

The one or more polypeptides may be present in nanoparticles or microparticles.

In one embodiment, the particles are biodegradable particles that may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The biodegradable particles may be prepared by methods known in the art. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, a particle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle may be a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle may comprise polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle may comprise a lipid, e.g., N-[1-(2,3-dioleoyloxy)propel]-N,N,N-trimethylammonium (DOTMA), 2,3-dioleyloxy-N-[2-spermine carboxamide]ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

A biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane](PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan.

In one embodiment, the microparticles have a diameter of about 1 to about 100 microns. In one embodiment, the diameter is about 1 to about 15 microns. In one embodiment, the diameter is about 5 to about 10 microns. In one embodiment, the diameter is about 15 to about 50 microns. In one embodiment, the diameter is about 20 to about 50 microns. In one embodiment, the diameter is about 100 to about 150 microns. In one embodiment, the diameter is about 500 to about 750 microns. In one embodiment, the diameter is about 150 to about 500 microns. In one embodiment, the diameter is about 200 to about 500 microns.

In one embodiment, the nanoparticles have a diameter of about 1 to about 100 nm. In one embodiment, the diameter is about 1 to about 15 nm. In one embodiment, the diameter is about 5 to about 10 nm. In one embodiment, the diameter is about 15 to about 50 nm. In one embodiment, the diameter is about 20 to about 50 nm. In one embodiment, the diameter is about 100 to about 150 nm. In one embodiment, the diameter is about 500 to about 750 nm. In one embodiment, the diameter is about 150 to about 500 nm. In one embodiment, the diameter is about 200 to about 500 nm.

In one embodiment, the protein(s) in the microparticle is/are released for up to 10 days. In one embodiment, the protein(s) in the microparticle is/are released for up to 14 days. In one embodiment, the protein(s) in the microparticle is/are released for up to 7 days. In one embodiment, the protein(s) in the microparticle is/are released for up to 4 days.

In one embodiment, the protein(s) in the microparticle begin being released at 2 hours. In one embodiment, the protein(s) in the microparticle begin being released at 4 hours. In one embodiment, the protein(s) in the microparticle begin being released at 8 hours. In one embodiment, the protein(s) in the microparticle begin being released at 12 hours.

### Pharmaceutical Compositions Comprising Cells and Delivery Thereof

The disclosure provides a composition comprising, consisting essentially of, or consisting of cells having a gene therapy vector and optionally a pharmaceutically acceptable (e.g., physiologically acceptable) carrier. When the composition consists essentially of the gene therapy vector containing cells, additional components can be included that do not materially affect the composition (e.g., adjuvants, buffers, stabilizers, anti-inflammatory agents, solubilizers, preservatives, etc.). When the composition consists of the gene therapy vector containing cells, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene therapy vector containing cells described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. Extemporaneous solutions and suspensions can be prepared from sterile powders, granules, and tablets of the kind previously described. In one embodiment, the carrier is a buffered saline solution.

In addition, one of ordinary skill in the art will appreciate that the gene therapy vector can be present in a cellular composition with other therapeutic or biologically-active agents. For example, factors that control inflammation, such as ibuprofen or steroids, can be part of the composition to reduce swelling and inflammation associated with in vivo administration of cells having the gene therapy vector. Immune system stimulators or inhibitors, or adjuvants, e.g., interleukins, lipopolysaccharide, and double-stranded RNA, can be administered. Antibiotics, i.e., microbicides and fungicides, can be present to treat existing infection and/or reduce the risk of future infection, such as infection associated with procedures.

Injectable depot forms are made by forming microencapsulated matrices with the cells in biodegradable polymers such as polylactide-polyglycolide. Depending on the ratio of cells to polymer, and the nature of the particular polymer employed, the rate of viral vector or VEGF release can be controlled. Examples of other biodegradable polymers include poly(orthoesters) and poly(anhydrides). Depot injectable formulations are also prepared by entrapping the cells in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene therapy vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene therapy vectors may be local using devices known in the art. Delivery may also be via surgical implantation of an implanted device.

The dose of the cells having the gene therapy vector in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the cells having the gene therapy vector described herein. A “therapeutically effective amount” refers to an amount effective, at dosages and for periods of time necessary, to achieve a desired therapeutic result. The therapeutically effective amount may vary according to factors such as the extent of vascularization desired, the void, deformity or defect to be treated, age, sex, and weight of the individual, and the ability of the gene therapy vector to elicit a desired response in the individual. The dose of gene therapy vector in the composition required to achieve a particular therapeutic effect typically is in units of vector genome copies per cell (gc/cell) or vector genome copies/per kilogram of body weight (gc/kg). One of ordinary skill in the art can readily determine an appropriate dose range for cells having a gene therapy vector, based on factors that are well known in the art. The therapeutically effective amount in the infected cells may be between 1×10¹⁰ genome copies to 1×10¹³ genome copies. The amount may be between 1×10¹¹ genome copies to 1×10¹⁴ genome copies. The amount may be between 1×10¹² genome copies to 1×10¹⁵ genome copies. The amount may be between 1×10¹³ genome copies to 1×10¹⁶ genome copies.

In one embodiment, the composition is administered single site of the mammal. It is believed that a single administration of the composition having the gene therapy vector results in persistent and optionally time limited expression of a proteins such as VEGF in the mammal with minimal negative side effects. However, in certain cases, it may be appropriate to administer the composition multiple times during a therapeutic period to ensure sufficient exposure of the composition. For example, the composition may be administered to the mammal two or more times (e.g., 2, 3, 4, 5, 6, 7, 8, 9, or 10 or more times) during a period to one or more sites in the body.

The present disclosure provides pharmaceutically acceptable compositions which comprise a therapeutically-effective amount of cells having a gene therapy vector comprising a nucleic acid sequence, e.g., one which encodes VEGF, an isoform thereof, or a portion thereof, the presence of which in transplanted cells in the mammal, increases blood vessel formation into the transplant and/or in tissues surrounding the transplant.

In particular, administration of the cells having a gene delivery vector in accordance with the present disclosure may be a singular occurrence at one body site, multiple occurrences at one body site, a singular occurrence at multiple body site or multiple occurrences at multiple body sites, depending, for example, upon the recipient's physiological condition, the desired result, and other factors known to skilled practitioners. Both local administration, e.g., at a surgery site, site of scarring and the like, and systemic administration, are contemplated. Any direct route of administration may be employed, e.g., injection at a site in need of therapy.

One or more suitable unit dosage forms comprising cells having the gene delivery vector(s), which may optionally be formulated for sustained release, can be administered by a variety of routes including, for example, administration to regions including rectal, buccal, vaginal, breast, buttocks, prostate, liver, lung, heart, pancreas, spleen, abdominal cavity, intestine, or fistulas. The formulations may, where appropriate, may include the step of bringing into association the vector or cell containing composition with liquid carriers, solid matrices, semi-solid carriers, finely divided solid carriers or combinations thereof, and then, if necessary, introducing or shaping the product into the desired delivery system.

The amount of cells having gene delivery vector(s) administered to achieve a particular outcome will vary depending on various factors including, but not limited to, the genes and promoters chosen, the condition, patient specific parameters, e.g., height, weight and age, the size of the void, defect or deformity, and the desired outcome.

Cells having the gene therapy vector may conveniently be provided in the form of formulations suitable for administration, e.g., via injection. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Cells having the gene therapy vector may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions of the disclosure can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors for delivery to cells can be provided in a dosage form containing an amount of a vector effective in one or multiple doses. For viral vectors, the dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10¹⁶ gc of viral vector can be nucleic acid or as a packaged virion. In some embodiments, about 10⁹ to about 10¹⁵ copies of viral vector, e.g., per 0.5 to 10 mL, can be employed as nucleic acid or as a packaged virion. The cells, nucleic acids or other vectors, can be employed in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount will vary depending on various factors including, but not limited to, the nucleic acid or vector chosen for administration, the disease, deformity, void or defect, the weight, the physical condition, the health, and/or the age of the mammal. Such factors can be readily determined by the clinician employing animal models or other test systems that are available in the art. As noted, the exact dose of cells having the gene therapy vector to be administered is determined by the attending clinician, but may be in 1 mL phosphate buffered saline. For delivery of plasmid DNA alone, or plasmid DNA in a complex with other macromolecules, the amount of DNA to be delivered to cells can vary. For example, from 0.0001 to 1 mg or more, e.g., up to 1 g, in individual or divided doses, e.g., from 0.001 to 0.5 mg, or 0.01 to 0.1 mg, of DNA.

For example, when a viral expression vector is employed, about 10⁸ to about 10⁶⁰ gc of viral vector can be employed as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the cells, nucleic acids or vectors, can be employed in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, administration of cells may be by injection or infusion using an appropriate catheter or needle. A variety of catheters may be used to achieve delivery, as is known in the art. For example, a variety of general purpose catheters, as well as modified catheters, suitable for use in the present disclosure are available from commercial suppliers.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

Pharmaceutical formulations containing the cells having the gene delivery vectors can be prepared by procedures known in the art using well known and readily available ingredients. For example, the vector can be formulated with common excipients, diluents, or carriers, and formed into tablets, capsules, suspensions, powders, and the like. The vectors of the disclosure can also be formulated as elixirs or solutions appropriate for parenteral administration, for instance, by intramuscular, subcutaneous or intravenous routes.

In one embodiment, the cells having the gene therapy vector may be formulated for administration, e.g., by injection, for example, bolus injection or continuous infusion via a catheter, and may include an added preservative.

These formulations can contain pharmaceutically acceptable vehicles and adjuvants which are well known in the prior art. It is possible, for example, to prepare solutions using one or more organic solvent(s) that is/are acceptable from the physiological standpoint.

The local delivery of the cells having the gene therapy vector can also be by a variety of techniques which administer the vector at or near the site of disease, defect, deformity or void, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### VEGF Formulations

Suitable compositions comprising cells having VEGF encoding vectors or comprising sustained release formulations comprising isolated VEGF polypeptide(s), e.g., in a microparticle or nanoparticle, include aqueous and non-aqueous solutions, isotonic sterile solutions, which can contain anti-oxidants, buffers, bacteriostats, and solutes that render the formulation isotonic with the blood or intraocular fluid of the intended recipient, and aqueous and non-aqueous sterile suspensions that can include suspending agents, solubilizers, thickening agents, stabilizers, and preservatives. Extemporaneous solutions and suspensions can be prepared from sterile powders, granules, and tablets of the kind previously described. When administering a composition, the pharmaceutically acceptable carrier may be a buffered saline solution.

In addition, the composition of the disclosure can comprise, or alternatively can be co-administered with, other therapeutic or biologically active agents. By “co-administration” is meant administration before, concurrently with, e.g., in combination with the composition in the same formulation or in separate formulations, or after administration of the composition as described above. For example, nucleic acid sequences, proteins, and/or other agents useful in the methods can be present and co-administered with the compositions of the disclosure. Suitable biologically active agents can include, for example, factors that control inflammation, such as ibuprofen or steroids, which can be co-administered to reduce swelling and inflammation associated with administration of the composition. Immunosuppressive agents can be co-administered to reduce inappropriate immune responses. Similarly, vitamins and minerals, anti-oxidants, and micronutrients can be co-administered. Antibiotics, e.g., microbicides and fungicides, can be co-administered to reduce the risk of infection.

Administration results in the subsequent release of VEGF. Selective isoform release may allow for an improved outcome. In one aspect of the disclosure, the nucleic acid molecule can be constructed by manipulating the splice donor, branch point, and/or splice acceptor regions. For example, a nucleic acid molecule can be included in an expression construct in cells and administered to a mammal to promote the production of VEGF189 at the expense of VEGF165 or VEGF121.

Suitable methods, both invasive and noninvasive methods, of directly administering the composition are available. Although more than one route can be used for administration, a particular route can provide a more immediate and more effective reaction than another route. The composition can be appropriately formulated and administered in the form of a local injection. The composition can be applied, for example, topically.

Local injections typically involve the administration of the composition by a catheter or needle. Pharmaceutically acceptable carriers for injectable compositions are well known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)).

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

In one embodiment, subjects include human subjects suffering from or at risk for the medical diseases and disorders described herein. The subject is generally diagnosed with the condition by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects, adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of treatment, as described above. In addition, the subject can have the genotype or phenotype for any hair color, eye color, skin color or any combination thereof. The term subject includes a subject of any body height, body weight, or any organ or body part size or shape.

The invention will be further described by the following non-limiting examples.

### Example 1

Fat augmentation is a widely used surgical procedure to provide volume for soft tissue reconstruction. It is generally carried out in 3 steps: lipoaspiration, e.g., from the abdomen or buttocks, concentration, and subsequent administration. The procedure is used for a variety of indications including but not limited to correction of congenital deformities, traumatic wounds, soft-tissue loss after oncologic surgery, skin grafting, scar and Dupuytren hand contracture, radiation damage, scleroderma deformations, and a variety aesthetic applications, e.g., augmentation of breast, face, buttock or hand.

Although autologous fat transplantation is a widely used surgical procedure to provide volume for soft tissue reconstruction, the long-term results are plagued by reabsorption rates of 30-70% within one year, primarily a result of ischemic necrosis of the transplanted adipocytes due to insufficient revascularization of the engrafted tissue.

In order to prolong the life of the fat transplant, obviating the need for repeated transplantation, in one embodiment, adenovirus-based gene therapy may be used to genetically modify harvested autologous fat prior to transplantation to induce vascularity into the transplanted fat from the tissues surrounding the transplant.

For example, following lipoaspiration and optional concentration, the fat is genetically modified using an adenovirus gene transfer vector to express at least one angiogenetic mediator. In one embodiment, the adenovirus vector expresses the angiogenic mediator 8 hours after administration, and then up to 1 to 2 additional weeks, in sufficient amounts to rapidly recruit vasculature into the transplanted fat from surrounding tissues, providing the vasculature for enhancing survival of the transplant.

### Example 2

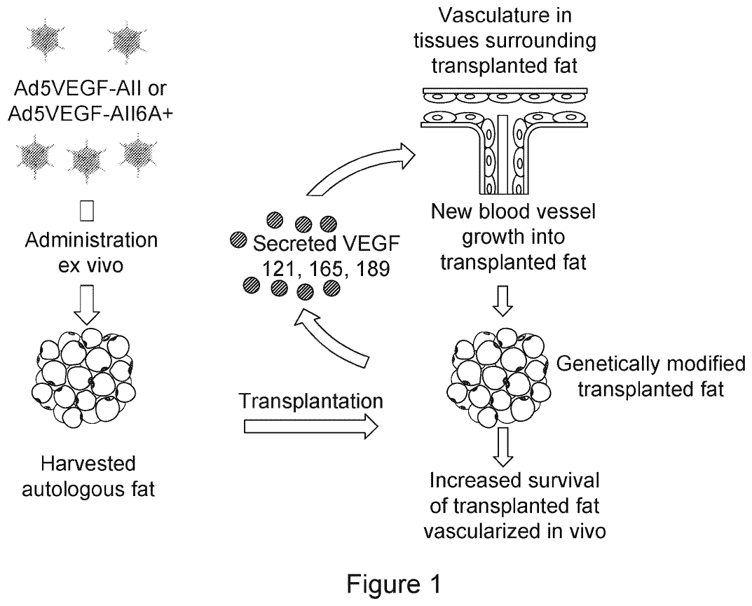

Since transplanted fat has no functional vasculature, the amount of transplant volume loss is high. Rapid establishment of a functioning vasculature provides a solution. Prior attempts to accomplish this have failed, likely because the angiogenic stimulus has not been sufficient in potency or availability to generate an effective vasculature from surrounding tissues. As discussed above, a strategy is to genetically modify the transplanted fat to secrete a powerful angiogenic mediator beginning as early as 8 hours after administration, and then for up to 1 to 2 additional weeks, in sufficient amounts to rapidly recruit vasculature into the transplanted fat from tissues surrounding the transplanted fat. In one embodiment, wan E1⁻E3⁻ serotype 5 adenovirus (Ad) gene transfer vector coding for all 3 major isoforms of VEGF is employed, genetically modifying autologous fat in the operating room prior to transplantation (FIG. 1). Once transplanted, the autologous genetically modified fat secretes all 3 VEGF major isoforms, thereby inducing vascularization into the transplanted fat from surrounding tissues.

Thus, the overall rationale is to promote rapid and robust angiogenesis by providing locally high concentrations of angiogenic mediators to the tissue to be vascularized (Baumgartner et al., (1998); Mack et al., (1998); Magovern et al., (1997); Rajagopalan et al., (2003); Rosengart et al., (1999); Rosengart et al., (1999); Takeshita et al., (1996); Takeshita et al., (1994); Freedman & Isner, (2002); Hershey et al., (2001); Khan et al., (2003); Shyu et al., (2003); Simovic et al., (2001)).

Ad-mediated expression of VEGF is effective in inducing angiogenesis in a variety of animal models (Mack et al., 1998; Magovern et al., 1997; Takeshita et al., 1996; Hershey et al., 2001; Ferrara et al., 2003; Neufeld et al., 1999; Robinson & Stringer, 2001; Tischer et al., 1991). VEGF consists of a family of proteins generated by alternative splicing of the primary RNA transcript (Ferrara et al., 2003; Neufeld et al., 1999; Robinson & Stringer, 2001; Tischer et al., 1991). After processing, the resulting major VEGF isoforms code for proteins of 121, 165 and 189 amino acids (Ferrara et al., 2003; Neufeld et al., 1999; Robinson & Stringer, 2001; Tischer et al., 1991). These VEGF isoforms are all angiogenic but differ in their biologic properties with respect to activation of various VEGF receptors and binding to extracellular matrix (Gitay-Goren et al., 1992; Park et al., 1993; Poltorak et al., 1997). Angiogenesis into the transplanted fat is induced from tissues surrounding the transplant by administering to the harvested fat, in one embodiment, an E1⁻E3⁻ adenovirus gene transfer coding for all 3 major isoforms of human VEGF. The Ad vectors may be either Ad5VEGF-AII (coding for VEGF 121, 165 and 189 in approximately equivalent amounts (see Whitlock et al. (2004), which is incorporated by reference herein) or AdVEGF-AII6A+ (coding for VEGF 121, 165 and 189 with amounts of 189>165>121; see Amano et al. (2005), which is incorporated by reference herein). The E1⁻E3⁻ Ad serotype 5 highly expresses the VEGF transgene in adipose tissue for approximately 2 weeks, which allows for induction of therapeutic angiogenesis, but not in excess. Because the Ad vector is delivered ex vivo to the harvested fat, preexisting anti-Ad5 antibodies, if present in the subject to be treated, should not interfere with efficacy of the induction of angiogenesis post-transplant.

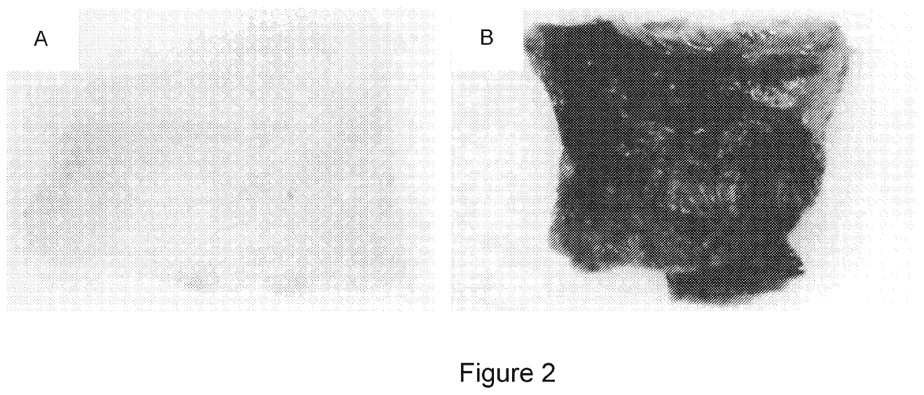

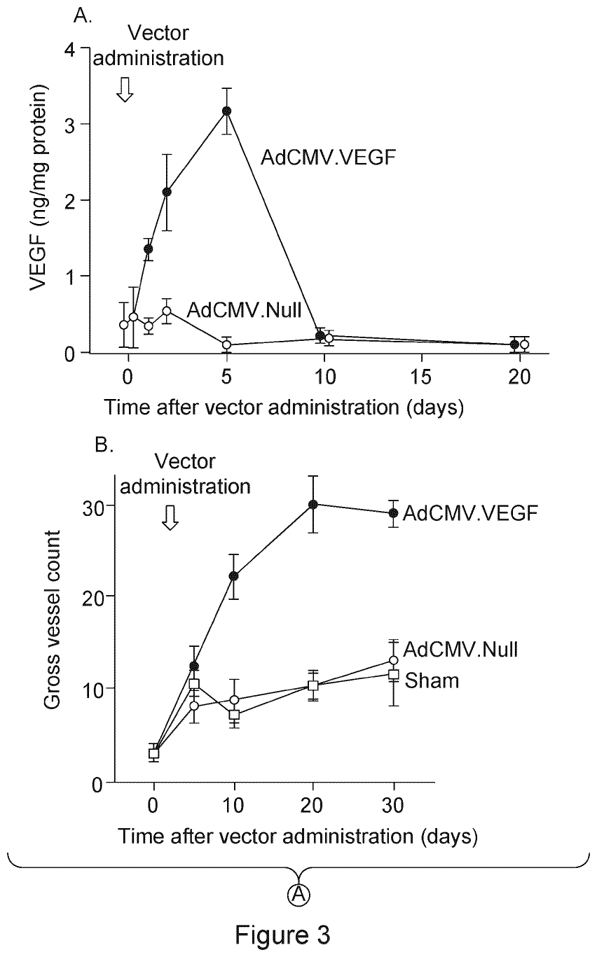

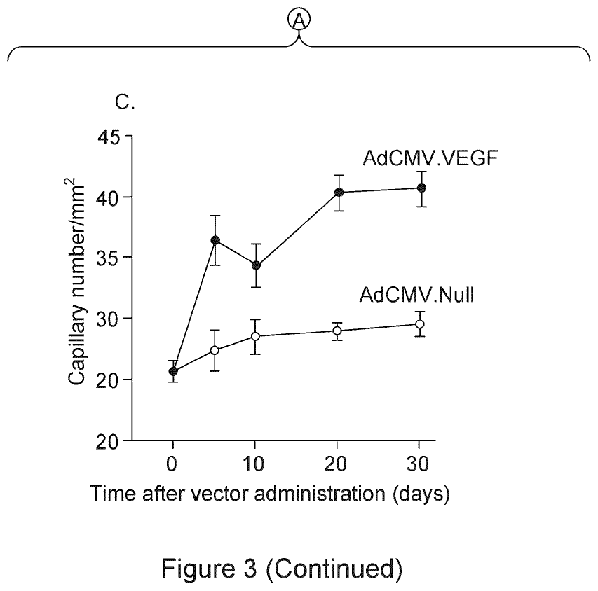

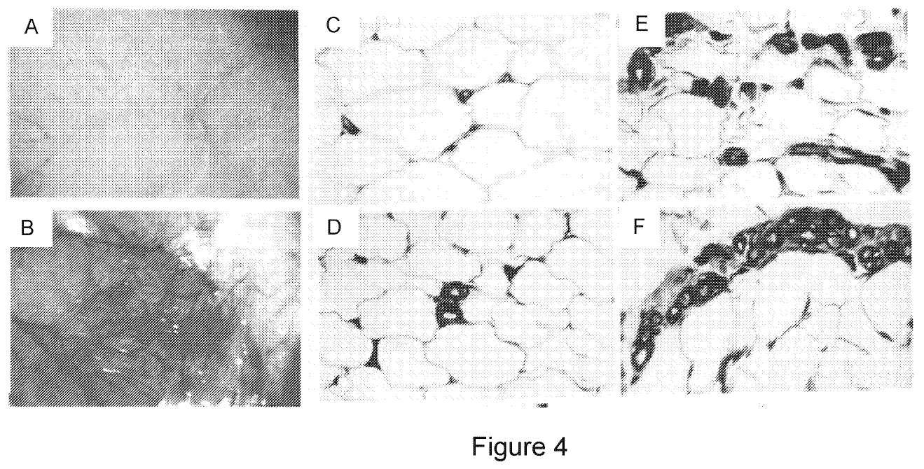

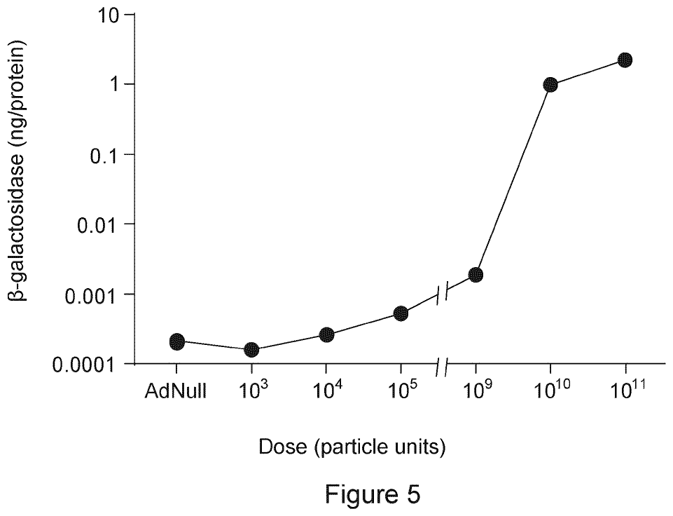

The data demonstrates that an Ad5 vector coding for a marker gene can effectively genetically modify adipose tissue (FIG. 2), an Ad5 vector coding for VEGF165 administered to fat can effectively express VEGF and induce vascularity (FIGS. 3 and 4) and that an Ad5 vector coding for a marker gene can effectively transfer and express a gene product in fat harvested from a human, similar to the fat used in transplantation (FIG. 5). Whitlock et al. and Amano et al. demonstrated in detail the effectiveness of the Ad5VEGF-AII and Ad5VEGF-AII6A+ vectors to induce angiogenesis. Ad5VEGF-AII and/or Ad5VEGF-AII6A+ are effective in improving survival of transplanted fat and Ad5VEGF-AII6A+ has been demonstrated to be safe in formal in vivo toxicology studies.

### Example 3

Autologous fat transplant for reconstructive surgery. Tissue damage resulting from traumatic injuries, surgical removal of tissue (for example, in oncology), surgical revision, or tissue damage due to radiation or fibrosis can benefit from autologous fat transplant with both structural and aesthetic benefits (Gir et al., 2012; Simonacci et al., 2017). Unfortunately, although initial results of fat transplants often meet expectations, fat transplants often exhibit reduced volume and plasticity over time resulting in disappointing results for long term applications (Leong et al., 2005; Locke et al., 2008; Kakagia et al., 2014; Bellini et al., 2017; Simonacci et al., 2017).

Angiogenesis as a potential mechanism to maintain the health and viability of transplanted fat. In autologous fat transplantation, the process of removing fat from one site and placing fat into a new site in the acceptor represents a traumatic manipulation of the fat tissue with loss of connections to blood vessels. In other clinical settings, enhanced angiogenesis has proven to result in improved tissue viability and survival. Optimal delivery of angiogenic biologics can be achieved by delivering the gene encoding an angiogenic factor into of the affected tissue or neighboring tissues to provide a constant, local source to enhance angiogenesis (Rosengart et al., 1999; Kalka et al., 2000; Vale et al., 2001; Rajagopalan et al., 2001; Mäkinen et al., 2002; Hedman et al., 2003; Mohler et al., 2003; Rajagopalan et al., 2003; Kim et al., 2004; Kalil et al., 2010; Kukula et al., 2011; Auger et al., 2013; Favaloro et al., 2013; Eibel et al., 2017; Hartikainen et al., 2017; Deev et al., 2018; Bard et al., 2021; Leikas et al., 2022; Povsic et al., 2023). Enhanced angiogenesis has been attempted as a means of improving the outcome of fat transplantation. Strategies have included vector-mediated gene delivery during transplantation, vector-mediated gene transfer to adipose-derived stem cells, or by co-delivery of endothelial cells (Yi et al., 2007; Lu et al., 2009; Jun-Jiang and Huan-Jiu, 2016; Dong et al., 2023), but to date, angiogenesis has not become an adjunct to fat transplantation in the clinic.

To enhance the likelihood of angiogenesis preserving fat tissue after transplantation, adenovirus-mediated expression of a chimeric VEGF gene that produces three alternatively-spliced isoforms of VEGF was employed, resulting in improved angiogenic performance (Whitlock et al., 2004; Amano et al., 2005). AdVEGFAII6A+ transduction of human fat may lead to a robust angiogenic response in the transplanted fat and improved retention of transplanted fat at late time points (3 to 6 months) after the procedure.

### Experimental Design

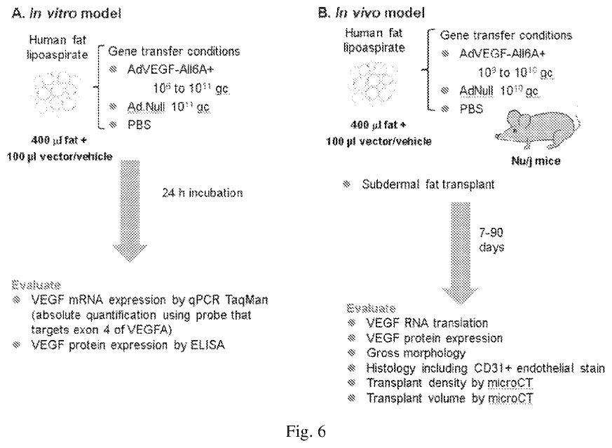

In vitro and in vivo models to assess the effect of gene transfer vectors on human fat lipoaspirates. A mouse model previously used by the Spector lab was employed to assess fat transplant (Dong et al., 2023). The model utilizes immunodeficient mice as recipients for a xenograft of fat obtained from human surgical specimens. The human fat undergoes minimal processing that includes mild tissue disruption as part of the lipoaspiration procedure used to collect the samples. The lipoaspirate was mixed with the adenoviral vectors expressing VEGF, control adenovirus vectors (Ad5Null), or PBS and then assessed in vitro or in vivo using a mouse model to determine the effect of VEGF gene transfer on the fat sample (FIG. 6). For the in vitro model, the fat is simple incubated for 24 hr before VEGF mRNA and protein expression are assessed. In the in vivo model, the human fat is transplanted subdermally in immunodeficient mice (Nu/j, Jackson Laboratory, Bar Harbor, ME, #002019) where it can be maintained indefinitely. Over time, transplants exhibit reduced volume and increased density signaling loss of fat cells and replacement by denser fibrotic structures. In an attempt to maintain the volume and fat content of the transplants, the fat is mixed with an adenoviral gene transfer vector (Ad5VEGFAII6A+) prior to transplant in the mouse at a dose ranging from 10⁹ to 10¹¹ genome copies (gc). Transgene expression is assessed at the level of RNA transcription via quantitative RT-PCR and at the protein level via an ELISA assay specific for human VEGF. Angiogenesis is evaluated in histological sections of the fat transplant using immunohistochemistry targeted to CD31 (PECAM), an endothelial cell surface protein. Both human and mouse CD31 are detected using indirect immunohistochemistry with diaminobenzidine (DAB) staining and a hematoxylin counterstain. CD3-positive staining is measured using QuPath image analysis software. Fat transplants are also assessed by microCT to evaluate the volume and density of the transplant.

### In Vitro Evidence for Gene Transfer

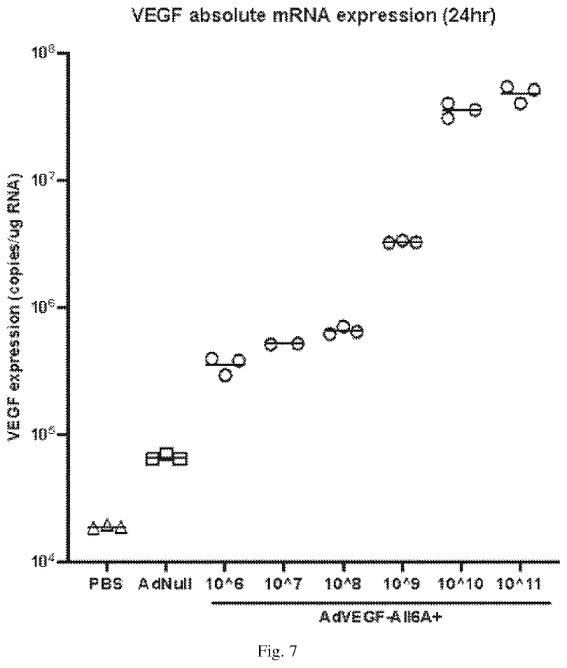

VEGF mRNA transcription. Using the in vitro model, samples of lipoaspirate treated with a range of concentrations of Ad5VEGFAII6A+ from 10⁶ gc to 10¹¹ gc were incubated at 37° C. for 24 hr. Following incubation, total mRNA was collected and compared to samples treated with Ad5Null (10¹⁰ gc) or PBS (vehicle). VEGF mRNA was quantified using RT-qPCR demonstrating a dose-response throughout the range of doses. Even the lowest dose (10⁶ gc) exhibited a significant increase in VEGF mRNA compared with the Ad5Null control treatment (10¹⁰ gc) (FIG. 7).

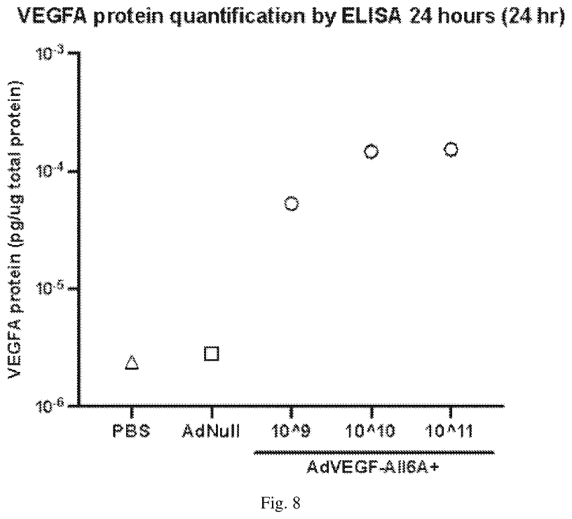

VEGF protein expression. Using the in vitro model, samples of lipoaspirate treated with a range of concentrations of Ad5VEGFAII6A+ from 10⁹ gc to 10¹¹ gc were incubated at 37° C. for 24 hr. Following incubation, VEGF protein concentration was assessed in a cell lysate prepared from the lipoaspirate-vector mixture and compared to samples treated with Ad5Null (10¹⁰ gc) or PBS (vehicle). VEGF protein was quantified using a commercially available enzyme-linked immunosorbent assay (ELISA). The analysis showed that Ad5VEGFAII6A+ yielded a dose-dependent increase in VEGF protein whereas treatment with Ad5Null (10¹⁰ gc) was not significantly different than PBS treatment (FIG. 8).

### In Vivo Evidence for Gene Transfer

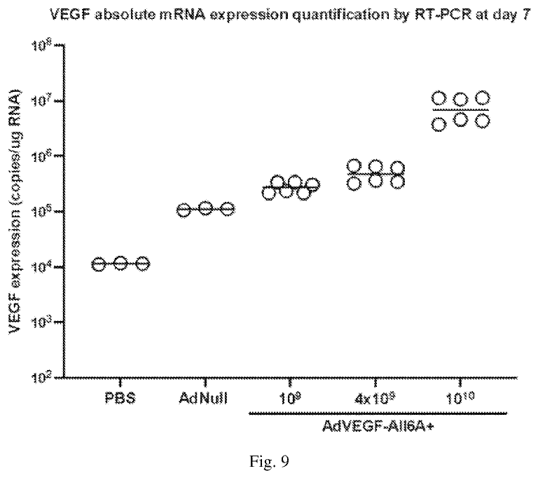

VEGF mRNA transcription. After demonstrating that the Ad5VEGFAII6A+ vector could transfer genes to cell in the lipoaspirate in vitro resulting in the production of VEGF mRNA and protein, the in vivo model was used to demonstrate that the lipoaspirate-vector mixture, once transplanted to the Nu/j mouse model, would also produce VEGF mRNA and protein in situ. Using the in vivo model described in FIG. 6, Nu/j mice received transplants of vector-treated human fat lipoaspirate after which mice were maintained for 7 days before recovery of the transplant and analysis of VEGF expression. Using three doses of vector separated by half logs (10⁹, 4×10⁹, and 10¹⁰ gc), analysis of mRNA from transplants using quantitative RT-qPCR revealed a dose-dependent increase in VEGF mRNA among samples receiving the Ad5VEGFAII6A+ vector while transplants treated with either 10¹⁰ gc Ad5Null or PBS contained significantly less VEGF mRNA (FIG. 9).

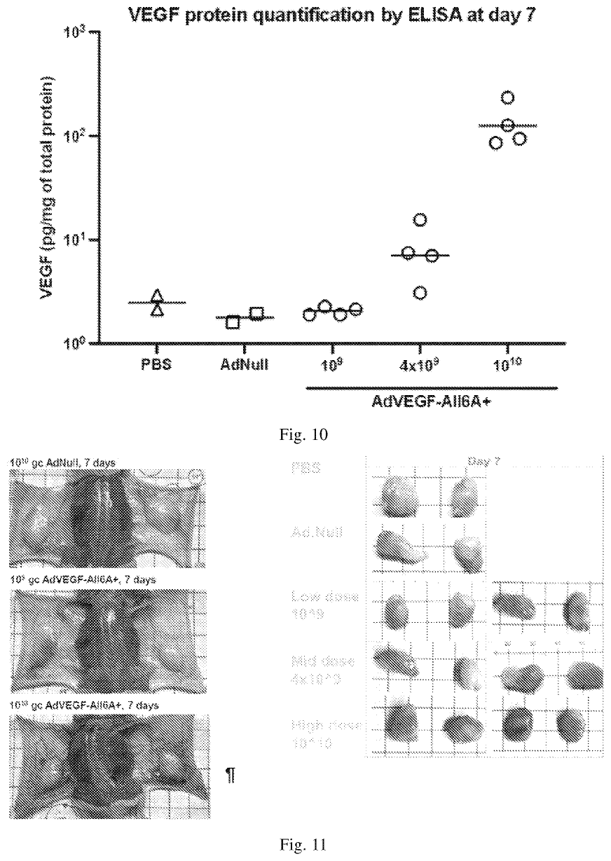

VEGF protein expression. Using the in vivo model described in FIG. 6, samples of lipoaspirate treated with Ad5VEGFAII6A+ at concentrations of 10⁹, 4×10⁹, or 10¹⁰ gc, Ad5Null at 10¹⁰ gc, or PBS. Mixtures were transplanted to Nu/j mice and were harvested 7 days later. VEGF protein concentration was assessed in a cell lysate prepared from the lipoaspirate-vector mixture. VEGF protein was quantified by ELISA. The analysis showed that Ad5VEGFAII6A+ produced a dose-dependent increase in VEGF protein. Notably, the lowest dose of the VEGF vector did not produce a significantly higher amount of VEGF than treatment with Ad5Null (10¹⁰ gc) or PBS (FIG. 10).

### In Vivo Evidence for Angiogenesis

Gross observation of VEGF-induced angiogenesis. In vivo experiments were designed to include Ad5VEGFAII6A+ doses ranging from 10⁹ to 10¹⁰ gc per transplant. Controls included Ad5Null at a concentration matching the highest dose of Ad5VEGFAII6A+ used in the experiment (10¹⁰ gc) and injection of vehicle (PBS) alone. Data was collected at 7, 14, 30, and 90 days after transplantation. When transplants were collected at 7 days post-transplant, the inner mucosal surface of the skin in mice that received high dose Ad5VEGFAII6A+(10¹⁰ gc) had an increase in both the number and diameter of large blood vessels in the skin in proximity to the transplant (FIG. 11, left). Lower doses of 4×10⁹ or 10⁹ gc had progressively less dermal blood vessels (i.e., the blood vessels associated with the inner aspect of the abdominal skin that has been dissected away from the body wall: “dermal blood vessels” are the blood vessels associated with the skin as opposed to the fat transplant), but both conditions had more dermal blood vessels at day 7 than mice bearing transplanted fat treated with Ad5Null (10¹⁰ gc). When fat transplants were removed on day 7, transplant-associated blood vessels could be observed (FIG. 11, right). The color of the vector-treated transplanted fat at day 7 was distinctly pink for most transplants, while fat transplants from either PBS- or Ad5Null-control animals were more likely to retain a yellow color.

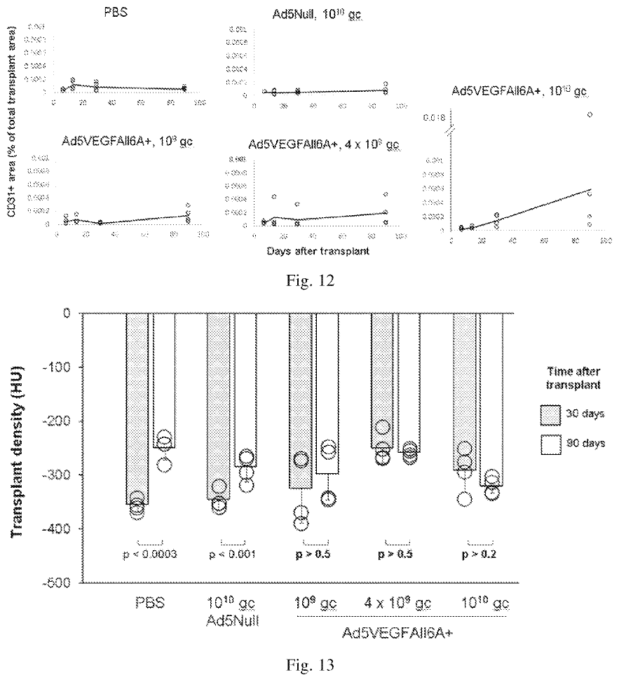

Enhanced angiogenesis observed using CD31 staining. To assess vascularization at the histological level, sections from the transplants were collected and stained for the presence of CD31, a marker of endothelial cells. The CD31 staining was detected with DAB which creates a brown-black stain on the tissue. The remaining structure of the tissue was revealed using a hematoxylin counter stain (light blue). QuPath image analysis software package was used to assess the area of CD31+ staining as a percent of the total transplant observed. The CD31+ staining area was calculated in transplants at days 7, 14, 30 and 90 days post-transplant. The data were expressed as CD31+ staining area as a percentage of total transplant area observed (FIG. 12). The area of tissue that stained positive for CD31 was largely unchanged from day 7 through day 90 for control conditions PBS and Ad5Null with all values for % CD31+ area falling below 0.0002% of total area. The low and middle doses of Ad5VEGFAII6A+ did not lead to significant increases in CD31+ area per transplant. In the high dose cohort, three of the four 90-day transplants had CD31+ percentage area above 0.0002% of total area with one transplant exhibiting CD31+ staining in approximately 0.018% of the total transplant area.

### In Vivo Impact of VEGF Gene Transfer on Transplanted Fat

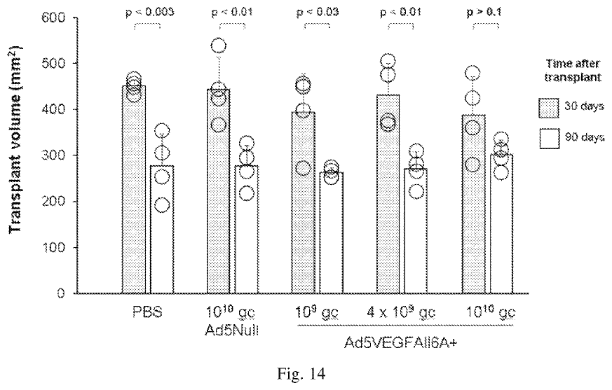

MicroCT assessment of transplant density and volume. At the time of sacrifice and prior to fixation, fat transplants were analyzed by microCT providing data on the density and volume of the transplanted fat. Fat density is an indicator of retained fat cells. Decreased density (lower value in Hounsfield Units) of the transplant is taken as an indication that the treatment has preserved fat cells or enhanced growth of new fat cells in the transplant (FIG. 13). When comparing transplant density at 30 days and 90 days post-transplant, transplants receiving control treatments (PBS or Ad5Null) both showed significant increases in density at 90 days compared with 30 days. In contrast, in all three conditions in which Ad5VEGFAII6A+ was administered, there was no significant increase in density between 30 and 90 days post-transplant. The mean value of the transplant density decreased in value for the two highest doses of Ad5VEGFAII6A+ suggesting that the trend in loss of the normal fat may have been prevented. Both control treatment conditions (PBS and Ad5Null) led to a significant loss of volume between days 30 and 90 post-transplant (FIG. 14). The two lower doses of Ad5VEGFAII6A+ also lost a significant amount of volume over the same time course. However, while transplants treated with the highest dose of Ad5VEGFAII6A+ did have a lower mean volume at 90 days compared with 30 days, the change in volume was not statistically significant suggesting that gene transfer of VEGF may stabilize transplant volume (FIG. 14).

### SUMMARY

The data showed that the VEGF-expressing vector is capable of producing VEGF-encoding RNA and VEGF protein in vitro after mixing the vector with the lipoaspirate and incubating for 24 hr. The data further showed that the VEGF-expressing Ad vector produced VEGF-encoding RNA and VEGF protein in vivo 7 days after delivery of a vector-treated fat transplant to an immune-compromised mouse. Expression of human VEGF in the mouse model correlated with the appearance of large blood vessels in the skin adjacent to transplants and on the surface of transplants that express high levels of VEGF. An increase area of immunohistochemical staining for CD31, a marker of endothelial cells, was observed 90 days after transplant placement, and at that 90 day timepoint, VEGF treated transplants had lower density than control transplants suggesting preservation of fat. While the volumes of treated and untreated transplants were not different at the 90 day timepoint, the trends suggest that a longer timepoint may reveal a positive result.

Thus, treatment of a fat lipoaspirate with an adenovirus gene transfer vector expressing a chimeric gene that produces multiple splice isoforms of human VEGF may enhance retention of fat transplants compared to untreated fat transplants or fat transplants treated with an adenovirus vector that does not express the VEGF gene.

### Example 4

### Exemplary Dose and Vessel Density

In one embodiment, a dose of 10¹⁰ gc Ad5VEGFAII6A+ in 100 ul PBS added to 400 ul lipoaspirate in mice resulted in a statistically significant elevation in VEGF mRNA and protein at day 7 after transplant compared with Ad5Null at the same concentration of vector (FIGS. 9 and 10). Based on an estimate for a minimal fat graft of about 0.1 milliliters and a fat density of about 0.9 g/ml (Fidanza, 2003), a dose in a human may be at least about 2.8×10⁹ gc in a 0.1 mL transplant.

Since the size of adipocytes changes after transplant due to the disruption of the tissue and prior vascularization, vascular density is inversely related to adipocyte size (Faber et al., 2012). Post-transplant, infected lipoaspirates may have a density of microvessels from about 30 to about 40 microvessels per mm² when measured in a paraffin tissue section using an endothelial stain (for example, Nakagawa et al., 2020).

### Example 5

### Exemplary Adenovirus Serotypes and Promoters

Adenovirus serotype 5 has an extensive safety record in humans. However, since >50% of adults have pre-existing anti-Ad5 neutralizing immunity (Nwanego et al., 2004; Abbink et al., 2007; Mast et al., 2010; Chen et al., 2010), vectors that have far less exposure in the human population may be desirable. For example, the chimpanzee adenovirus serotype AdC7 has been shown to have low pre-existing neutralizing titers in human populations (<5% in the USA; <15% in Africa; Chen et al., 2010). In one embodiment, an Ad5 vector is employed for gene delivery, e.g., in Ad5 seronegative individuals. In one embodiment, an AdC7 vector is employed for gene delivery.

With regard to promoters, in one embodiment strong viral promoters that are expressed work in most human cells, such as the CMV promoter, strong constitutive eukaryotic promoters such as EF1a or chicken beta-actin promoters, optionally coupled with viral enhancers such as the CAG promoter that couples the chicken beta-actin promoter with the CMV enhancer, or cell-specific promoters potentially including adipocyte-specific promoters like the adiponectin promoter or endothelial-specific promoters such as the VE-cadherin promoter may be employed.

### REFERENCES

Abbink et al., J. Virol., 81:4654 (2007).
Amano et al., Mol. Ther., 12:716 (2005).
Auger et al., Annu. Rev. Biomed. Eng., 15:177 (2013).
Bard et al., J. Cardiovasc. Transl. Res., 14(3):409 (2021).
Baumgartner et al., Circulation., 97:1114 (1998).
Bellini et al., Ann. Med. Surg. (Lond)., 24:65 (2017).
Cao et al., Clin. Plast. Surg., 50:171 (2023).
Charles-de-S A et al., Plast. Reconstr. Surg., 135:999 (2015).
Chen et al., J. Virol., 84:10522 (2010).
Deev et al., Ther. Adv. Cardiovasc. Dis., 12(9):237 (2018).
Dong et al., Cells Tissues Organs, _:_ (2022).
Dong et al., Cells Tissues Organs, 212:341 (2023).
Dong et al., Cells Tissues Organs, 212:341-351 (2023).
Eibel et al., Cytokine, 91:44 (2017).
Faber et al., Int. J. Obes., 36:1078 (2012).
Favaloro et al., Catheter Cardiovasc Interv., 82(6):899 (2013).
Ferrara et al., Nat. Med., 9:669 (2003).
Fidanza, F., Acta Diabetol., 40 (Suppl 1), s242 (2003).
Firriolo et al., Plast. Reconstr. Surg., 150:1340e (2022).
Fontes et al., Ann. Med. Surg. (Lond)., 36:212 (2018).
Freedman & Isner, Ann. Intern. Med., 136:54 (2002).
Gause et al., Adipocyte, 3:273 (2014).
Gir et al., Plast. Reconstr. Surg., 130:249 (2012).
Gitay-Goren et al., J. Biol. Chem., 267:6093 (1992).
Gontijo-de-Amorim et al., Aesthet. Surg. J., 37:975 (2017).
Gontijo-de-Amorim et al., Aesthet. Surg. J., 38:NP37 (2018).
Hamed et al., PLoS One, 5:e13986 (2010).
Harris et al., Aesthet. Surg. J., 39:220 (2019).
Hartikainen et al., Eur. Heart. J., 38(33):2547 (2017).
Hedman et al., Circulation, 107(21):2677 (2003).
Hershey et al., Cardiovasc. Res., 49:618 (2001).
Hu et al., Aesthetic. Plast. Surg., 46:2218 (2022).
Jiang et al., Plast. Reconstr. Surg., 150:578 (2022).
Jun-Jiang et al., Artif. Cells Nanomed. Biotechnol., 44:1141 (2016).
Kakagia & Pallua, Surg. Innov., 21:327 (2014).
Kalka et al., Circ. Res., 86(12):1198 (2000).
Kaminsky et al., Hum. Gene Ther. Clin. Dev., 24:38 (2013).
Khan et al., Gene. Ther., 10:285 (2003).
Khouri & Khouri, Plast. Reconstr. Surg., 140:466e (2017).
Kim et al., Exp. Mol. Med., 36(41:336 (2004).
Kølle et al., Stem Cells Transl. Med., 9:1277 (2020).
Leikas et al., J. Gene. Med., 4(8):e3437 (2022).
Leong et al., J. Dermatol. Sci., 37:169 (2005).
Liu et al., Aesthetic Plast. Surg., 47:808 (2023).
Liu et al., Aesthetic. Plast. Surg., _:_ (2022).
Locke & de Chalain, Ann. Plast. Surg., 60:98 (2008).
Lu et al., Plast. Reconstr. Surg., 124:1437 (2009).
Mack et al., J. Vasc. Surg., 27:699 (1998).
Magovern et al., Hum. Gene. Ther., 8:215 (1997).
Major et al., Trends Biotechnol., 40:77 (2022).
Mäkinen et al., Mol. Ther., 6(1):127 (2002).
Malik et al., Plast. Reconstr. Surg. Glob. Open., 8:e2835 (2020).
Mast et al., Vaccine, 28:950 (2010).
Mohler et al., Vasc. Med., 8(1:9 (2003).
Nakagawa et al., Breast Cancer, 27:77 (2020).
Neufeld et al., Faseb i., 13:9 (1999).
Nolan et al., Adipocyte., 10:80 (2021).
Nwanegbo et al., Clin. Diagn. Lab. Immunol., 11:351 (2004).
Park et al., Mol. Biol. Cell., 4:1317 (1993).
Poltorak et al., J. Biol. Chem., 272:7151 (1997).
Povsic et al., Circ. Cardiovasc. Interv., 16(8):e012997 (2023).
Rajagopalan et al., Circulation, 104(7):753 (2001).
Rajagopalan et al., Circulation, 108(16):1933 (2003).
Robinson & Stringer, J. Cell. Sci., 114:853 (2001).
Rosengart et al., Ann. Surg., 230:466 (1999).
Rosengart et al., Circulation, 100:468 (1999).
Shauly et al., Plast. Reconstr. Surg. Glob. Open, 10:e3987 (2022).
Shih et al., Semin. Plast. Surg., 34:5 (2020).
Shyu et al., Am. J. Med., 114:85 (2003).
Simonacci et al., Ann. Med. Surg. (Lond)., 20:49 (2017).
Simovic et al., Arch. Neurol., 58:761 (2001).
Swanson, Aesthet. Sura. J., 38:NP34 (2018).
Takeshita et al., Biochem. Biophys. Res. Commun., 227:628 (1996).
Takeshita et al., J. Clin. Invest., 93:662 (1994).
Tischer et al., J. Biol. Chem., 266:11947 (1991).
Toyserkani et al., Stem Cells Transl. Med., 6:1666 (2017).
Vale et al., Circulation, 103(17):2138 (2001).
Whitlock et al., Mol. Ther., 9:67 (2004).
Yi et al., J. Plast. Reconstr. Aesthet. Sura., 60:272 (2007).
Zhao et al., Asian J. Sura. 46:35 (2023).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to induce blood vessel formation after transplant in a mammal, comprising:
providing isolated mammalian tissue comprising fat cells and optionally stromal vascular fraction (SVF) from the mammal;
infecting the tissue with an adenovirus expressing one or more isoforms of VEGF; and
transplanting at least a portion of the infected tissue into the mammal so as to induce neovascularization at, into or near the site of transplant.

2. The method of claim 1 wherein the mammal is a human.

3. The method of claim 1 wherein the tissue comprises mammalian adipocytes or adipose stem cells, or both.

4. The method of claim 1 wherein the adenovirus expresses two or more isoforms of VEGF, expresses VEGF 121, expresses VEGF165, expresses VEGF189 or expresses VEGF121, VEGF165 and VEGF189.

5-8. (canceled)

9. The method of claim 4 wherein the ratio of the isoforms is about 1:1:1 or wherein the ratio of the isoforms is VEGF189>VEGF165>VEGF121.

10-12. (canceled)

13. The method of claim 1 wherein the adenovirus genome or capsid is from a human adenovirus or a non-human primate adenovirus.

14-18. (canceled)

19. The method of claim 1 wherein the fat tissue is obtained from a thigh, abdomen, intra-abdominal, or buttock of the mammal or at least a portion of the infected tissue is transplanted to a breast, buttock, intra-abdominal, intra-thoracic, face, scar, contracture, or hand of the mammal.

20. (canceled)

21. A method, comprising:
providing isolated mammalian tissue comprising fat cells; and
infecting the tissue with an amount of an adenovirus expressing one or more isoforms of VEGF.

22. The method of claim 21 wherein the mammal is a human.

23. The method of claim 21 wherein the amount is at least 10¹¹ genome copies.

24. The method of claim 21 wherein the tissue comprises mammalian adipocytes or adipose stem cells, or both.

25. The method of claim 21 to wherein the adenovirus expresses two or more isoforms of VEGF, the adenovirus expresses VEGF121, the adenovirus expresses VEGF165, the adenovirus expresses VEGF189 or the adenovirus expresses VEGF121, VEGF165 and VEGF189 optionally in a 1.1:1 ratio or the ratio of the isoforms is VEGF189>VEGF165>VEGF121.

26-33. (canceled)

34. The method of claim 21 wherein the adenovirus is a human adenovirus or a non-human primate adenovirus.

35-36. (canceled)

37. The method of claim 1 wherein the adenovirus is serotype 5, 6, 26, 35 or 36.

38. The method of claim 1 wherein the dose of the adenovirus is at least 2×10⁹, 5×10⁹ or 1×10¹⁰ gc/0.1 mL.

39. Isolated infected mammalian fat tissue prepared by the method of claim 21.

40. (canceled)

41. The isolated mammalian fat tissue of claim 39 which is at least 0.1 g to 1 g, 1 g to 10 g, 10 g to 25 g, 25 g to 50 g, 50 g to 100 g, or 100 g to 500 g.

42. The isolated mammalian fat tissue of claim 39 which has at least 1×10⁴ to 1×10⁶ cells, at least 1×10⁶ to 1×10⁸ cells, least 1×10⁸ to 1×10¹⁰ cells, least 1×10¹⁰ to 1×10¹² cells, least 1×10¹² to 1×10¹⁴ cells or least 1×10¹⁴ to 1×10¹⁶ fat cells.

43. A method to augment a void, defect or deformity, augment soft tissue, enhance split or full thickness grafts, alter scarring or augment the volume or shape of a body part in a mammal, comprising: administering to a mammal in need thereof a composition comprising isolated mammalian fat tissue infected with an adenovirus expressing one or more isoforms of VEGF.

44-45. (canceled)

46. The method of claim 43 wherein the mammal has congenital deformities, traumatic wounds, soft-tissue loss after oncologic surgery, is in need of skin grafting, has one or more scars, has Dupuytren hand contractures, has radiation damage, has deformation from scleroderma, has tendinopathies, or has arthritis.

47-51. (canceled)


SEQUENCE LISTING (39 sequences)

SEQ ID NO: 1 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 2 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 4-9 - skipped (6 entries)

SEQ ID NO: 10 - protein, 171 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 11 - protein, 209 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 12 - DNA, 444 bp, Homo sapiens
  source 1..444: mol_type = other DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtga

SEQ ID NO: 13 - DNA, 516 bp, Homo sapiens
  source 1..516: mol_type = other DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtatgtgacaagccgaggcggtga

SEQ ID NO: 14 - DNA, 576 bp, Homo sapiens
  source 1..576: mol_type = other DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 15 - DNA, 630 bp, Homo sapiens
  source 1..630: mol_type = other DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 16 - DNA, 648 bp, Homo sapiens
  source 1..648: mol_type = other DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 17 - DNA, 699 bp, Homo sapiens
  source 1..699: mol_type = other DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 18-20 - skipped (3 entries)

SEQ ID NO: 21 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 22 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 23 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 24 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 25 - protein, 211 aa, Homo sapiens [sequence omitted]

SEQ ID NO: 26 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 27 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 28 - protein, 353 aa, Homo sapiens [sequence omitted]

SEQ ID NO: 29 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 30 - protein, 728 aa, Homo sapiens; identical to SEQ ID NO: 29

SEQ ID NO: 31 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 32 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 33 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 34 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 35 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 36 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 37 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 38 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 39 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI